(19) United States
(12) Patent Application Publication
(10) Pub. No.: US 2026/0240693 A1
(43) Pub. Date: Aug. 20, 2026

(54) A DISPOSABLE ABSORBENT PANT-TYPE ARTICLE AND A METHOD FOR PRODUCING A DISPOSABLE ABSORBENT PANT-TYPE ARTICLE

(71) Applicant: Essity Hygiene and Health Aktiebolag, Göteborg (SE)

(72) Inventors: Lucas BÄCK, Göteborg (SE); Hans EEN, Göteborg (SE); Rickard LÖNNROTH, Göteborg (SE)

(73) Assignee: Essity Hygiene and Health Aktiebolag, Göteborg (SE)

(21) Appl. No.: 19/489,523

(22) PCT Filed: Jun. 13, 2023
(86) PCT No.: PCT/EP2023/065835

§ 371 Date: Dec. 2, 2025

(51) Int. Cl. A61F 13/496; A61F 13/15; A61F 13/49

(52) U.S. Cl. CPC A61F 13/496; A61F 13/15593; A61F 13/49012; A61F 13/49019; A61F 13/4902; A61F 2013/49025; A61F 2013/49033; A61F 2013/49038

(57) ABSTRACT

The present disclosure relates to a disposable pant-type article having a front body panel, a rear body panel, and a crotch region. An absorbent assembly including an absorbent core is arranged with a central part located in the crotch region. The front body panel is joined to the rear body panel by first and second front panel side edges being joined to first and second rear panel side edges in first and second side seams, respectively, thus forming a waist opening and leg openings. The front or rear body panel is a combined panel including first and second panel portions connected by a connection region. The first and second panel portions are bonded by first and second bottom layers being bonded to first and second top layers for attaching sets of elastomeric strands therebetween.

### TECHNICAL FIELD

The present disclosure pertains to a disposable absorbent pant-type article comprising: a front body panel and, a rear body panel, and a crotch region located between the front body panel and the rear body panel. An absorbent core is arranged with a central part of the absorbent core in the crotch region. A first front panel side edge is joined to a first rear panel side edge in a first side seam and a second front panel side edge is joined to a second rear panel side edge in a second side seam thereby forming a waist opening and two leg openings in the disposable pant-type article. The disclosure further pertains to a continuous method of producing elastic disposable pant-type articles. The disclosure also pertains to a combined laminate material web for a disposable hygienic article, and to a method for producing a combined laminate material web.

### BACKGROUND

In the art of disposable pant-type articles and in particular disposable pant-type articles which are intended for use by adult users, the articles should meet high demands on fit, comfort and have a tailored and underwear like appearance while at the same time meeting equally high demands on absorbency and leakage protection. Disposable absorbent articles are articles which are intended to be discarded after a single use and not be laundered or otherwise restored for repeated use.

Pant-type articles of this kind are relatively complex products involving multiple components which are combined in high-speed processes to form the absorbent article. Such components include nonwoven components, barrier components, elastic components and usually an absorbent core which may be provided in the form of an absorbent assembly including a barrier layer and a liquid permeable topsheet which together enclose the absorbent core.

Threaded elastic laminates, formed by sandwiching pre-tensioned elastomeric threads between layers of web materials, e.g., non-woven web materials, are used to create pant-type articles with body-conforming properties resembling those of high-quality textile underwear. In addition, it is common to enhance fit and leakage security by arranging pre-tensioned elastic elements along all or selected parts of the waist opening and leg openings of the pant-type article.

Numerous methods are known for providing threaded elastic laminates. Adhesive bonding methods involve application of adhesive to bond the pre-tensioned elastomeric threads between the layers of web materials.

Welding bonding methods involve the application of a weld bond pattern to bond the pre-tensioned elastomeric threads between the layers of web materials. The weld bonds may be produced by heat bonding or ultrasonic bonding.

In view of sustainability requirements as well as economic requirements, welding bonding methods may be preferred since such methods do not require the application of an adhesive. However, in in-line continuous production, welding bonding methods have been found to be difficult to adapt to achieve secure attachment of pre-tensioned elements that have curved shapes, such as may be the case for pre-tensioned elastic elements used e.g., along all or selected parts of the leg openings of the pant-type articles.

Moreover, regardless of the bonding method used, requirements for breathability, adaptability and softness of the threaded laminates imparts other requirements to the bonding method, e.g., regarding the size and distribution of the bonds.

Accordingly, there exists an ongoing need for improved or alternative threaded elastic laminates in a disposable absorbent patent-type article and for improved or alternative methods for forming such a threaded elastic laminate.

### SUMMARY

In a first aspect, the need may be fulfilled by an absorbent article according to claim 1.

As such, there is provided a disposable pant-type article, the article having a longitudinal direction and a transverse direction, and defining a thickness direction perpendicular to the longitudinal direction and the transverse direction, wherein an inside of the article is towards the wearer when the article is worn, and an outside of the article is away from the wearer when the article is worn, the article comprising:
a front body panel,
a rear body panel, and
a crotch region being located between the front body panel and the rear body panel, wherein an absorbent assembly comprising an absorbent core is arranged with a central part of the absorbent core being located in the crotch region.

The front body panel is joined to the rear body panel by a first front panel side edge being joined to a first rear panel side edge in a first side seam, and a second front panel side edge being joined to a second rear panel side edge in a second side seam, such that the article forms a waist opening, defined by a waist edge of the front panel and a waist edge of the rear panel, and a pair of leg openings, defined by a pair of leg edges of the front body panel and a pair of leg edges of the rear body panel.

Each of the front body panel and the rear body panel has a top side and a bottom side as seen in the thickness direction of the article.

Herein, the terms “top side” and “bottom side” do not refer to any specific orientation in the thickness direction of the article (unless otherwise stated). Instead, the top side of a body panel is one out of the inside and the outside of the body panel, and the bottom side of a body panel is the outer one out of the inside and the outside of the body panel.

At least one of the front body panel and rear body panel is a combined panel, which combined panel comprises a pair of leg elastic members arranged adjacent the pair of leg edges of the combined panel, wherein each leg elastic member extends at least partially in a direction other than parallel with the transversal direction. Further, the combined panel comprises, as seen along the longitudinal direction, a first panel portion and a second panel portion, the first panel portion and the second panel portion being connected by a connection region. The first panel portion extends from the connection region and in the longitudinal direction towards the waist edge of the combined panel, and the second panel portion extends from the connection region and in a longitudinal direction towards the pair of leg edges of the combined panel. The second panel portion comprises the pair of leg elastic members.

The first panel portion comprises a first bottom layer and a first top layer being superimposed such that, in the first panel portion, the first bottom layer forms a bottom side surface of the combined panel and the first top layer forms a top side surface of the combined panel. The first panel portion further comprises a first set of elastomeric strands being sandwiched between the first bottom layer and the first top layer, the elastomeric strands being aligned substantially in parallel with each other and with the transversal direction, and the elastomeric strands being spaced apart from each other in the longitudinal direction. The first panel portion is bonded by the first bottom layer being bonded to the first top layer by a welded bond pattern attaching the first set of elastomeric strands between the first bottom layer and the first top layer.

The second panel portion comprises a second bottom layer and a second top layer being superimposed such that, in the second panel portion, the second bottom layer forms a bottom side surface of the combined panel and the second top layer forms a top side surface of the combined panel. The pair of leg elastic members are sandwiched between the second bottom layer and the second top layer, and the second panel portion is bonded by the second bottom layer being bonded to the second top layer by adhesive bonds, attaching the pair of leg elastic members between the second bottom layer and the second top layer.

Optionally, the second panel portion further comprises a second set of elastomeric strands, wherein the elastomeric strands are aligned substantially in parallel with each other and with the transversal direction, and the elastomeric strands are spaced apart from each other in the longitudinal direction. In this case, the second panel portion is bonded by the second bottom layer being bonded to the second top layer by adhesive bonds, attaching the pair of leg elastic members and the second set of elastomeric strands between the second bottom layer and the second top layer.

By virtue of the combined panel comprising a first panel portion which is bonded by welded bond pattern and a second panel portion which is bonded by adhesive, it is possible to provide a front or rear body panel which is bonded by adhesive where this is required to enable attachment of the leg elastic members, where each leg elastic member extends at least partially in a direction other than parallel with the transversal direction. Thus, by using a welded bonding pattern in a first panel portion and an adhesive bonding pattern in a second panel portion, secure attachment of curved or angled leg elastic members may be achieved without sacrificing breathability and flexibility of the panel material in areas other than those where the leg elastic members are applied.

The first panel portion comprises no elastic members being substantially inclined in relation to the transversal direction T, and can thus suitably be bonded by a welded bond pattern.

In each of the first panel portion and the second panel portion, the respective top and bottom layers forms a top side surface and a bottom side surface, respectively, of the combined panel. Accordingly, in each of the first panel portion and the second panel portion, the combined panel comprises no additional layers of web material but the top and bottom layers. A front or rear body panel having relatively few layers is beneficial in terms of breathability and comfort as well as in view of sustainability and economics.

A top side surface is a surface forming part of the top side of the panel, i.e. forming part of the inside or the outside of the panel, depending on the orientation of the panel in the article.

A bottom side surface is a surface forming part of the bottom side of the panel, i.e. forming part of the inside or the outside of the panel, depending on the orientation of the panel in the article.

In each of the first and second panel portions, the first set of elastomeric strands and the optional second set of elastomeric strands, respectively, are attached between the respective top and bottom layer when in a stretched condition. Accordingly, in a relaxed state of the combined panel, the respective top and bottom layers form wrinkles along the sets of elastomeric strands. The combined panel is extendible from the relaxed state to a stretched-out state in which the sets of elastomeric strands are stretched out along the transversal direction T causing the wrinkles of the combined panel to diminish or to completely flatten out.

The first panel portion, the second panel portion, and the connection region are transversal portions of the combined panel, and extend from the first side edge of the combined panel to the second side edge of the combined panel.

For example, the first panel portion, the second panel portion and the connection region may each be a transversal portion or region of the panel. In other words, the first panel portion, the second panel portion, and the connection region may each have a continuous length in the longitudinal direction as seen over the entire width of the combined panel in the transverse direction.

That each leg elastic member extends at least partially in a direction other than parallel with the transversal direction implies that the leg elastic member is at least partially curved or inclined such that it spans over a longitudinal extension as seen in the longitudinal direction. Each leg elastic member may for example form an angle of at least 10 degrees with the transversal direction, over at least a portion of the longitudinal extension, such as over at least 20% of the longitudinal extension of the leg elastic member. For example, each leg elastic member may from an angle of from 10 to 80 degrees with the transversal direction, over at least a portion of the longitudinal extension of the leg elastic member, such as over at least 20% of the longitudinal extension of the leg elastic member.

For example, each leg elastic member may span over a longitudinal extension as seen in the longitudinal direction of at least 50 mm.

For example, each leg elastic member may form an angle of at least 10 degrees with at least one strand in the second set of elastomeric strands. Preferably, each leg elastic member may form an angle of at least 10 degrees, such as from 10 to 80 degrees, with a majority of the strands in the second set of the elastomeric strands.

Each leg elastic member may hence be a curved leg elastic member such as is generally known in the art of absorbent articles.

The welded bond pattern of the first panel portion may comprise pairs of adjacent weld bonds for attaching each of the elastomeric strands of the first set using such pairs of adjacent weld bonds.

The welded bonds are preferably arranged such that the elastomeric strands are mechanically trapped between the weld bonds. For example, each of the elastomeric strands of the first set may be mechanically trapped between the adjacent weld bonds of a plurality of pairs of adjacent weld bonds. Thus, the first panel portion may be free or at least substantially free from adhesive.

Optionally, the adhesive bonds of the second panel portion are applied to one or more out of the second top layer, the second bottom layer, the optional second set of elastomeric strands, and the leg elastic members.

Optionally, in the second panel portion, each leg elastic member crosses at least one elastic strand out of the second set of elastomeric strands.

In some variants, the connection region D may be infinitely narrow as seen along the longitudinal direction, i.e., the connection region may for example be in the form of a transversely extending line connecting the first panel portion and the second panel portion.

In other variants, the connection region may have an extension in the longitudinal direction of the article, for example the connection region may comprise a joint joining the first panel portion and the second panel portion.

Such a joint joining the first panel portion and the second panel portion may for example be an adhesive joint or a welded joint.

Optionally, the connection region has a length in the longitudinal direction of the article being no more than 10 mm.

Optionally, the connection region has a length in the longitudinal direction of the article being no more than 20% of a total length (in the longitudinal direction of the article) of the combined panel.

Preferably, the connection region has a length in the longitudinal direction of the article being no more than 10 % of a total length (in the longitudinal direction of the article) of the combined panel, such as no more than 7% or no more than 5% of a total length (in the longitudinal direction of the article) of the combined panel.

Optionally, the first panel portion has a length in the longitudinal direction of the article, the length being at least 20 % of a total length of the combined panel, such as at least 25 %, or at least 30%. For example, the first panel portion may have a first length being less than 65 % of a total length of the combined panel in the longitudinal direction of the article. For example, the first panel portion may have a length in the longitudinal direction of the article being from 25 % to 65% of the total length of the combined panel.

Optionally, the second panel portion has a length in the longitudinal direction of the article being at least 20%, such as at least 25% or at least 30% of a total length of the at least one of the combined panel in the longitudinal direction of the article. For example, the second panel portion may have a length being less than 70% of the total length of the combined panel. For example, the second panel portion may have a length being from 25 % to 70 % of the total length of the combined panel.

Optionally, the second bottom layer and the first bottom layer are formed by sheets of identical sheet material. As such, the bottom side of the combined panel may have a uniform appearance.

Optionally, the second bottom layer and the first bottom layer are formed by a single continuous material sheet, extending over the connection region. As such, the bottom side of the combined panel may have a uniform appearance without any visible joints at the connection region. Also, in such variants, the connection region may comprise no more than three layers of sheet material.

The first panel portion and the second panel portion each comprises no more than two layers of sheet material-this being the result of the respective top layer and bottom layer each forming one out of a top side surface and a bottom side surface of the combined panel in the first panel portion and the second panel portion, and that no intervening layer of sheet material is arranged between the top layer and the bottom layer.

It is to be understood that when it is referred herein to “a top layer being bonded to a bottom layer”, this implies that the top layer is directly bonded to the bottom layer, without any intervening layers of sheet material. However, this does not exclude that the elastic strands and/or any leg elastic members could be involved in the bonding.

Thus, optionally, the first panel portion and the second panel portion each comprises no more than two layers of sheet material and the connection region comprises no more than three layers of sheet material.

Further, as will be explained below, using a single continuous material sheet to form the second bottom layer and the first bottom layer may be advantageous in manufacturing.

Also, when the second bottom layer and the first bottom layer are formed by a single continuous material sheet, and the bottom side of the combined panel is the outside of the absorbent article, this means that the outside surface of the absorbent article is formed by the single continuous material sheet of the combined panel, thus providing the article with a uniform outside appearance without any visible joints.

Optionally, the second top layer and the first top layer are formed by separate material sheets.

Optionally, the second top layer and the first top layer are formed by separate sheets of identical sheet material, such as by separate sheets of the same sheet material.

Optionally, in the connection region, a transversal edge portion of the material sheet of the second top layer is arranged so as to overlap a transversal edge portion of the material sheet of the first top layer in the longitudinal direction, preferably the transversal edge portion of the second top layer is arranged on the top side of the transversal edge portion of the first top layer.

As such, the connection region may have a length in the longitudinal direction as proposed above, where the length of the connection region in the longitudinal direction corresponds to the length in the longitudinal direction of the longitudinal overlap between the first top layer and the second top layer. Optionally, the top direction is directed towards the inside of the article and the bottom direction is directed towards the outside of the article.

Optionally, the adhesive bonds of the second panel portion comprises, in defined regions overlapping the leg elastic members, a first adhesive applied to one of the second top layer and the second bottom layer, and a second adhesive applied to the other one of the second top layer and the second bottom layer, wherein optionally the second adhesive is different from the first adhesive.

Optionally, the first adhesive is applied substantially continuously over the second top layer.

Optionally, the second adhesive is applied only in defined regions of the second bottom layer overlapping the leg elastic members.

Optionally, the first adhesive is more fast consolidating than the second adhesive.

Optionally, the adhesive bonds of the second panel portion comprises adhesive applied to the second set of elastic strands.

Optionally, the combined panel further comprises a waist panel portion comprising the waist edge of the combined panel.

Optionally, the combined panel defines a transversal waist connection region. In this case the first panel portion extends in the longitudinal direction from the connection region to the waist connection region, and the waist panel portion extends in the longitudinal direction from the waist connection region to the waist edge of the combined panel.

Optionally, the waist panel portion comprises a waist bottom layer and a waist top layer and a third set of elastomeric strands being sandwiched between the waist bottom layer and the waist top layer, the elastomeric strands being aligned substantially in parallel with each other and with the transversal direction, and the elastomeric strands being spaced apart from each other in the longitudinal direction. The waist panel portion is bonded by the waist bottom layer being bonded to the waist top layer by a welded bond pattern attaching the third set of elastomeric strands between the waist bottom layer and the waist top layer

The waist bottom layer forms a bottom side surface of the combined panel, and the waist top layer forms a top side surface of the combined panel.

Optionally, a length of the waist panel portion in the longitudinal direction of the article is at least 5%, such as from 5% to 20%, or from 5% to 15% of a total length of the combined panel in the longitudinal direction of the article.

Optionally, the waist bottom layer and the waist top layer may be formed from the same sheet material being folded along the waist edge.

Optionally, the waist bottom layer and the first bottom layer may be formed by a single continuous material sheet, extending over the transversal waist connection region.

In some variants, the second bottom layer, the first bottom layer, and the waist bottom layer may be formed by a single continuous material sheet, extending over the connection region and the waist connection region.

The waist top layer and the first top layer may each be formed by a separate material sheet, wherein the material sheets are arranged with a longitudinal overlap in the waist connection region.

In some variants, a single continuous material sheet may form the first bottom layer, the waist bottom layer and the waist top layer, wherein the waist bottom layer and the waist top layer are separated by a fold in the single continuous material sheet extending along the waist edge.

As used herein, the term “layer” refers to a layer in a laminate material or in a portion of a laminate material, which layer comprises a sheet formed material or a portion of a sheet formed material. A “layer” can be bonded to another layer by means of weld bonds or adhesive bonds as disclosed herein, sandwiching elastic strands or members between the layers.

The sheet formed material may be a sheet of any suitable material, such as of a nonwoven material. Optionally, at least one, preferably each, of the second bottom layer, the second top layer, the first bottom layer, the first top layer, and optionally the waist bottom layer, and the waist top layer comprises or consists of a sheet of a nonwoven material.

Optionally, the nonwoven material has a basis weight of from 8 to 26 gsm.

Optionally, the nonwoven material contains thermoplastic fibers, preferably the nonwoven material contains at least 50% thermoplastic fibres and more preferably at least 80% thermoplastic fibres.

Optionally, the second set of elastomeric strands and/or the first set of elastomeric strands and/or optionally the third set of elastomeric strands has a linear mass density, dtex, of from 80 to 1200 dtex. Optionally, the average spacing in the longitudinal direction between the strands in the second set of elastomeric strands of the second panel portion is no less than the average spacing in the longitudinal direction between the strands in the first set of elastomeric strands in the first panel portion.

Optionally, the spacing in the longitudinal direction between the strands in the first set of elastomeric strands of the first panel portion is from 2 to 20 mm. For example, the spacing between the strands in the first set of elastomeric strands may be constant.

Optionally, the spacing in the longitudinal direction between the strands in the second set of elastomeric strands of the second panel portion is from 2 to 35 .mm. For example, the spacing between the strands in the first set of elastomeric strands may be constant.

Optionally, the spacing in the longitudinal direction between the strands in the third set of elastomeric strands of the waist portion is from 2 to 12 mm. For example, the spacing between the strands in the first set of elastomeric strands may be constant.

A set of elastic strands as referred to herein comprises a plurality of elastic strands.

For example, the first and/or the second set of elastic strands may each comprises a plurality of elastic strands, i.e. no less than two strands. For example, the first and/or second set of elastic strands may each comprise no less than three strands, such as no less than four strands or no less than five strands.

For example, the third set of elastic strands (of the waist portion) may comprise a plurality of elastic strands, such as no less than two strands or no less than three strands.

Optionally, the absorbent core is arranged with a central part of the absorbent core being located in the crotch region and with a front end part of the absorbent core overlapping with the front body panel in a front overlap area and with a rear end part of the absorbent core overlapping with the rear body panel in a rear overlap area.

Optionally, the absorbent core is arranged so as to longitudinally overlap a transversal portion of the connection region of the combined panel.

Optionally, the front body panel is a combined panel and the rear body panel is a combined panel.

This implies that the article is provided with leg elastic members in both the front body panel and the rear body panel.

Optionally, one of the front body panel and the rear body panel is a combined panel (i.e. comprises leg elastic members), and the other of the front body panel and rear body panel is free from leg elastic members.

Optionally, one of the front body panel and the rear body panel is a combined panel (i.e. comprises leg elastic members), and the other of the front body panel and rear body panel is free from adhesive.

Optionally, said other of the front body panel and the rear body panel comprises a main panel portion comprising a main bottom layer and a main top layer and a set of elastomeric strands being sandwiched between the main bottom layer and the main top layer, the elastomeric strands being aligned substantially in parallel with each other and with the transversal direction, and the elastomeric strands being spaced apart from each other in the longitudinal direction.

The main portion is bonded by the main bottom layer being bonded to the main top layer by a welded bond pattern.

Optionally, the main panel portion extends at least from the leg edges of said other of the front body panel or rear body panel, respectively and in a direction towards the waist edge of said other of the front body panel and rear body panel.

As mentioned in the above, the front body panel and the rear body panel of the article extends from a first side edge to a second side edge. As such, the front body panel and/or the rear body panel may each consist of a piece of a laminate material which extends continuously from the first side edge to the second side edge.

For example, the combined panel may consist of a piece of combined laminate material which is cut from a combined laminate material web, as will be described in the below.

Hence, in a second aspect, the disclosure relates to a combined laminate material web according to claim 46. The combined laminate material of the second aspect may be used in the pant-type disposable article as disclosed herein.

Thus, there is provided a combined laminate material web for a disposable hygienic article, the combined laminate material web being continuous as seen in a machine direction and having a width in a cross-direction, perpendicular to the machine direction, between two side edges. The combined laminate material web has a top side and a bottom side as seen in a thickness direction perpendicular to the machine direction and to the cross-direction. The combined laminate material web comprises a first web portion and a second web portion, the first web portion and the second web portion being connected by a connection region extending along the machine direction, such that the first web portion extends from the connection region and in the cross-direction towards the first side edge of the combined laminate material web, and the second web portion extends from the connection region and in the cross-direction towards the second side edge of the combined laminate material web.

The first web portion comprises a first bottom layer and a first top layer being superimposed such that, in the first web portion, the first bottom layer forms a bottom side surface and the first top layer forms a top side surface of the combined laminate material web. The first web portion further comprises a first set of elastomeric strands being sandwiched between the first bottom layer and the first top layer, the elastomeric strands being aligned substantially in parallel with each other and with the machine direction, and the elastomeric strands being spaced apart from each other in the cross-direction, and the first web portion is bonded by the first bottom layer being bonded to the first top layer by a welded bond pattern attaching the first set of elastomeric strands between the first bottom layer and the first top layer, and the second web portion comprises a second bottom layer and a second top layer being superimposed such that, in the second web portion, the second bottom layer forms a bottom side surface and the second top layer forms a top side surface of the combined laminate material web. The second web portion further comprises additional elastic members extending at least partially in a direction other than parallel with the machine direction, wherein the additional elastic members are sandwiched between the second top layer and the second bottom layer. The second web portion is bonded by the second bottom layer being bonded to the second top layer by adhesive bonds, for attaching the additional elastic members between the second bottom layer and the second top layer.

Optionally, the second web portion further comprises a second set of elastomeric strands being sandwiched between the second bottom layer and the second top layer, the elastomeric strands being aligned in parallel with each other and substantially with the machine direction, and the elastomeric strands being spaced apart from each other in the cross-direction. In this case, the second web portion is bonded by the second bottom layer being bonded to the second top layer by adhesive bonds, for attaching the additional elastic members and the second set of elastomeric strands between the second bottom layer and the second top layer.

As such, a front body panel or a rear body panel being a combined panel as described herein may be formed by cutting such a combined material web. Thus, it will be understood that to form a combined panel including any of the optional features as set out herein in relation to the article of the first aspect, corresponding features may be applied to the combined laminate material web of the second aspect. In a third aspect, there is provided a method for forming a combined laminate material web in accordance with claim 26.

Thus, there is provided a method for forming a combined laminate material web for a disposable hygienic article, the combined laminate material web being continuous as seen in a machine direction and having a width in a cross-direction perpendicular to the machine direction between a first side edge and a second side edge, the combined laminate material web having a top side and a bottom side as seen in a thickness direction perpendicular to the machine direction and to the cross-direction. The method comprises a welding bonding step and an adhesive bonding step.

The welding bonding step comprises feeding a first bottom layer web, a first top layer web, and a first set of continuous elastic strands in the machine direction, and bonding the first bottom layer web to the first top layer web by a welded bond pattern so as to attach the first set of continuous elastic strands between the first bottom layer web and the first top layer web, the first set of elastomeric strands being aligned in parallel with each other and substantially with the machine direction, and being spaced apart from each other in the cross-direction.

The adhesive bonding step comprise feeding a second top layer web, a second bottom layer web, in the machine direction, and feeding a set of one or more continuous additional elastic members, and bonding the second bottom layer web to the second top layer web by adhesive bonds so as to attach the second set of elastomeric strands and the set of one or more continuous additional elastic members between the second bottom layer web and the second top layer web. The additional elastic members are extending at least partially in a direction not parallel with the machine direction.

Optionally, the adhesive bonding step further comprises feeding a second set of continuous elastic strands in the machine direction, and bonding the second bottom layer web to the second top layer web by adhesive bonds so as to attach the second set of elastomeric strands and the set of one or more continuous additional elastic members between the second bottom layer web and the second top layer web. The second set of elastomeric strands are being aligned substantially in parallel with each other and with the machine direction, and being spaced apart from each other in the cross-direction.

The additional elastic members extending at least partially in a direction not parallel with the machine direction may form the leg elastic members in an article comprising a combined panel obtained from the combined laminate material web.

That the additional elastic members extend at least partially in a direction other than parallel with the machine direction implies that each additional elastic member is at least partially curved or inclined such that it spans over a CD extension as seen in the cross-direction. Each additional elastic member may for example form an angle of at least 10 degrees with the machine direction, over at least a portion of the CD extension, such as over at least 20% of the CD extension of the additional elastic member. For example, each additional elastic member may from an angle of from 10 to 80 degrees with the machine direction, over at least a portion of the CD extension of the additional elastic member, such as over at least 20% of the CD extension of the additional elastic member.

For example, each additional elastic member may span over a CD extension as seen in the longitudinal cross-direction of at least 50 mm.

For example, each additional elastic member may form an angle of at least 10 degrees with at least one strand in the second set of elastomeric strands. Preferably, each additional elastic member may form an angle of at least 10 degrees, such as from 10 to 80 degrees, with a majority of the strands in the second set of the elastomeric strands.

The welding bonding step and the adhesive bonding step are performed so as to form a first web portion, wherein the first bottom layer web forms a bottom side surface and the first top layer web forms a top side surface of the combined laminate material web, and a second web portion, wherein the second bottom layer web forms a bottom side surface and the second top layer web forms a top side surface of the combined laminate material web. The second web portion is connected to the first web portion via a connection region extending along the machine direction.

Herein, “top” and “bottom” does not relate to any specific vertical orientation of the combined laminate material web, only to relative locations as seen along a thickness direction perpendicular to the cross-direction and the machine direction.

The welding bonding step and the adhesive bonding step are performed such that the first and second sets of elastomeric strands are attached between the respective top and bottom layers when in a stretched state in relation to the respective top and bottom layers. For example, the elastomeric strands may be stretched so as to be elongated to a length being at least 2× a relaxed length of the elastomeric strands, such as to a length being at least 3× a relaxed length of the elastomeric strands. The respective top and bottom layers may be stretched only slightly, i.e., to an amount required to perform the feeding operation. As such, the elastomeric strands are attached when in a stretched state in relation to the respective top and bottom layer.

Optionally, the welding bonding step is performed upstream of the adhesive bonding step in the machine direction. As such, it may be avoided that any adhesive, such as non-consolidated adhesive, risks adhering to the welding equipment used for the welding bonding step, and thereby deteriorate its function and/or require cleaning.

Optionally, in the adhesive bonding step, the elastomeric strands of the second set are aligned substantially in parallel with each other and with the machine direction, and the additional elastic members are extending at least partially in a direction not parallel with the machine direction, such that each additional elastic member crosses at least one elastic strand out of the second set of elastomeric strands.

Optionally, the welded bond pattern in the welding bonding step comprises pairs of adjacent weld bonds for attaching each of the first set of elastomeric strands using such pairs of adjacent weld bonds, as disclosed herein.

Optionally, the adhesive bonds of the adhesive bonding step are applied to one or more of the second top layer, the second bottom layer, the second set of elastomeric strands, and the leg elastic members.

Optionally, the connection region has a width in the cross-direction being no more than 10 mm.

Optionally, the connection region has a width in the cross-direction being no more than 20% of a total width of the combined laminate material web, such as no more than 10% of a total width of the combined laminate material web. For example, the connection region may have a width in the cross-direction being no more than 7% or no more than 5% of a total width of the combined laminate material web.

Optionally, the first web portion has a width in the cross-direction being at least 20% of a total width in the cross-direction of the combined laminate material web, such as at least 25%, or at least 30%. For example, the first web portion may have a width in the cross-direction being less than 65% of a total width of the combined laminate material web. For example, the first panel portion may have a width in the cross-direction being from 25% to 65% of the total width of the combined laminate material web. Optionally, the second web portion has a width in the cross-direction being at least 20% of a total width in the cross-direction of the combined laminate material web, such as at least 25%, or at least 30%. For example, the second web portion may have a width in the cross-direction being less than 70% of a total width of the combined laminate material web. For example, the second panel portion may have a width in the cross-direction being from 25% to 70% of the total width of the combined laminate material web.

Optionally, the welding bonding step comprises
feeding a combined bottom layer web material to form the first bottom layer web, and bonding the first top layer web to a first width portion of the combined bottom layer web material so as to form the first laminate web portion, leaving at least a second width portion of the combined bottom layer web material non-laminated, and
the adhesive bonding step comprises feeding the combined bottom layer web material to form the second bottom layer web, and
bonding the second top layer web to the non-laminated second width portion of the combined bottom web so as to form the second laminate web portion.

Optionally, the second top layer web and the first top layer web are fed as separate web materials.

Optionally, the second top layer web and the first top layer web are formed by webs of the same material.

Optionally, the method comprises comprising feeding a base top layer web in the machine direction, and slitting the base top layer web along the machine direction so as to form the second top layer web and the first top layer web as separate webs from the base top layer web.

Optionally, the adhesive bonding step comprises bonding the second top layer web in a partially overlapping relationship with the first top layer web, as seen along the cross-direction, such that the connection region comprises the overlapping portions of the second top layer web and the first top layer web.

As set out in the above, the method may involve feeding a combined bottom layer web material to form the first bottom layer web and the second bottom layer web, such that the combined bottom layer web material extends over the connection region. Thus, the connection region may be formed so as to comprise no more than three layers of sheet material.

Thus, optionally, the first panel portion and the second panel portion are formed to comprise no more than two layers of sheet material (the respective top and bottom layers) and the connection region is formed so as to comprise no more than three layers of sheet material.

When the method involves feeding a combined bottom layer web material to form the first bottom layer web and the second bottom layer web, such that the combined bottom layer web material extends over the connection region, the first top layer web and the second top layer web could optionally be applied so as to be spaced in the cross-direction on the combined bottom layer web. In such a case, the connection region would comprise only one layer of sheet material, namely the combined bottom layer web material.

In another option, the first top layer web and the second top layer web could be applied edge-to-edge as seen in the cross-direction on the combined bottom layer web. In such a case, the connection region would comprise only the line where the first top layer web and the second top layer web meet. In this case, the number of layers in the connection region will be no more than two.

Optionally, the adhesive bonding step comprises, in defined regions overlapping the leg elastic members, applying a second adhesive applied to one of the second top layer and the second bottom layer, and a first adhesive applied to the bottom one of the second top layer and the second bottom layer, wherein optionally the second adhesive is different from the first adhesive.

Optionally, the second adhesive is applied substantially continuously over the second bottom layer.

Optionally, the first adhesive is applied only in defined regions of the second top layer overlapping the additional elastic members.

Optionally, the second adhesive is more fast consolidating than the first adhesive.

Optionally, the adhesive bonding step comprises applying adhesive to the second set of elastic strands.

Optionally, at least one, preferably each, of the second bottom layer web, the second top layer web, the first bottom layer web, the first top layer web, and optionally the waist bottom layer web, and the waist top layer web comprises or consists of a sheet of a nonwoven material.

Optionally, the nonwoven material has a basis weight of from 8 to 26 gsm.

Optionally, the nonwoven material contains or consists of thermoplastic fibers, preferably the nonwoven material contains contain at least 50% thermoplastic fibres and more preferably at least 80% thermoplastic fibres.

Optionally, the second set of elastomeric strands and/or the first set of elastomeric strands and/or optionally the third set of elastomeric strands has a linear mass density, dtex, of from 80 to 1200 dtex.

Optionally, the average longitudinal spacing between the strands in the second set of elastomeric strands is no less than the average longitudinal spacing between the strands in the first set of elastomeric strands.

Optionally, the longitudinal spacing between the strands in the first set of elastomeric strands is from 2 to 20 mm.

Optionally, the longitudinal spacing between the strands in the second set of elastomeric strands is from 2 to 35 mm.

Optionally, the method further comprises a waist forming step comprising forming a waist laminate web portion of the combined laminate material web, the waist laminate web portion comprising a side edge of the combined laminate material web.

Optionally, the waist laminate web portion is connected to the first laminate web portion via a waist connection region.

Optionally, the waist forming step comprises feeding a waist bottom layer web, a waist top layer web, and a third set of elastomeric strands in the machine direction, and
bonding the top waist layer web to the bottom waist layer web by a welded bond pattern attaching the third set of elastomeric strands between the waist bottom layer web and the waist top layer web,
wherein the elastomeric strands are aligned substantially in parallel with each other and with the machine direction, and the elastomeric strands are spaced apart from each other in the cross-direction.

Optionally, the waist forming step is performed upstream of the adhesive bonding step, and preferably downstream of the welding bonding step.

Optionally, in the waist laminate web portion, the waist bottom layer web forms a bottom side surface and the waist top layer web forms a top side surface of the combined laminate material web.

Optionally, the waist laminate web portion has a width in the cross-direction being at least 5%, such as from 5% to 10% or from 5% to 10% of the width of the total width of the combined laminate material web.

Optionally, the combined laminate material web is for forming a combined panel being a front body panel or a rear body panel of a disposable pant-like article, wherein the additional elastic members are applied so as to form leg elastic members in the front body panel or rear body panel.

Optionally, the method may further comprise cutting or deactivating the additional elastic members so as to form sets of elastic members separated by a non-elastic portion, where the non-elastic portion is intended to connect to the crotch section of the absorbent article. The sets of additional elastic members thus formed may in turn be cut when forming front or rear body panels out of the continuous material web, such that each set forms one member of a pair of leg elastic members on a first panel, and one member of a pair of leg elastic members on a second panel.

A combined laminate material web produced by the method of the third aspect is encompassed by the present disclosure. In particular, a combined laminate material web according to the second aspect, comprising the features as obtained by any of the variants of the method for producing a combined laminate material web according to the third aspect is encompassed herein.

In a fourth aspect there is provided a method for producing a disposable pant-type article comprising providing a combined laminate material web according to the second aspect.

Thus, there is provided a method for producing a disposable pant-type article comprising providing a combined laminate material web in accordance with the method of the third aspect, and forming a disposable pant-type article having a front body panel and a rear body panel, a crotch region being located between the front body panel and the rear body panel;
an absorbent assembly comprising an absorbent core being arranged with a central part of the absorbent core being located in the crotch region a second front panel side edge being jointed to a second rear panel side edge in a second side seam; and a first front panel side edge being joined to a first rear panel side edge in a first side seam, wherein at least one out of the front body panel and rear body panel is formed from the combined laminate material web.

Optionally, the method for producing a disposable pant-type article comprises providing a rear body panel web and a front body panel web, at least one out of the rear body panel web and the front body panel web being a combined laminate material web produced in accordance with the third aspect described in the above, and intermittently applying absorbent assemblies to the rear body panel web and the front body panel web, the absorbent assemblies being applied spaced-apart in the machine direction and being attached with a first end part of the core assembly overlapping the rear body panel web and a second end part of the core assembly overlapping the front body panel web, so as to form a composite web of interconnected precursor articles each precursor article comprising an absorbent assembly. The composite web of precursor articles has a front waist edge and a rear waist edge. The method comprises folding the composite web of interconnected precursor articles along a fold line extending in the machine direction and bringing the rear waist edge of the rear body panel web into alignment with the front waist edge of the front body panel web, forming first and second side seams in each precursor article, the side seams joining the front body panel web with the rear body panel web and being formed in the cross-direction along dividing lines between the precursor articles; and severing individual pant-type articles from the composite web of precursor articles.

Optionally, the top side of the combined laminate web is towards the inside of the article and the bottom side of the combined laminate web is towards the outside of the article.

Optionally, the method comprises arranging the absorbent core with a central part of the absorbent assembly being located in the crotch region and with a front end part of the absorbent core overlapping with the front body panel web in a front overlap area and with a rear end part of the absorbent core overlapping with the rear body panel web in a rear overlap area.

Optionally, the method comprises arranging the absorbent assembly so as to overlap at least a part of the first web portion.

Optionally, the front body panel web is formed from a second combined laminate material web and the rear body panel web is formed from a first combined laminate material web, each of the second and first combined laminate material webs being formed by the method according to the third aspect in the above.

Optionally, one of the front body panel and the rear body panel is formed from a combined laminate material web being formed by the method according to the third aspect in the above, and the other of the front body panel and rear body panel is free from leg elastic members forming extending at least partly in a direction other than parallel with the machine direction.

Optionally, one of the front body panel and the rear body panel is formed from a combined laminate material web being formed by the method according to the third aspect in the above, and the other of the front body panel and rear body panel is free from adhesive.

The front body panel may be formed from a continuous front body panel web which extends transversely across the width of the pant-type article between the second and the first front/rear panel side edges and/or the elastically stretchable rear body panel may be formed from a continuous rear body panel web which extends transversely across the width of the pant-type article between the second and the first rear panel side edges.

In the pant-type disposable articles as disclosed herein, the absorbent core may form part of an absorbent assembly, the absorbent assembly comprising the absorbent core, a liquid permeable topsheet and a liquid impermeable backsheet, wherein the absorbent core is sandwiched between the liquid permeable topsheet and the liquid impermeable backsheet.

The disposable pant-type articles as disclosed herein may comprise an elastic waist feature. The elastic waist feature may form a continuous waistband encircling the waist opening of the disposable pant-type article, or may be applied only to selected portions of the waist opening, such as only along central portions of the waist edge of the front and rear body panels or only along the waist edge of the rear body panel, etc.

The elastic waist feature may be completely overlapping with the front body panel and/or with the rear body panel and may share a waist edge with the corresponding front body panel and/or rear body panel.

The elastic waist feature may be completely overlapping with the front body panel and/or with the rear body panel or may be applied to a waist edge of the front body panel and/or rear body panel and protrude beyond the waist edge of the corresponding front body panel and/or rear body panel.

The elastic waist feature may comprise one or more elastic elements. The waist feature may comprise a plurality of elastic strands or elastic bands. Alternatively, the elastic waist feature may comprise or consist of a band of elastic material, such as elastic nonwoven, elastic foam or elastic film. The elastic waist feature may be attached to the front body panel and/or to the rear body panel as a prefabricated component, such as a waistband component comprising two layers of carrier web with elastic strands or bands attached between the layers of carrier web. An elastic waist feature comprising elastic elements such as strands or bands may alternatively be formed directly on the elastic body panel material by applying the elastic elements to the body panel along a waist edge of the body panel and subsequently covering the elastic elements by a cover web, such as a nonwoven cover web.

Optionally, in some variants, the elastic waist feature may be a component of the disposable pant-type article which is distinct from the body panel material of the front body panel and the rear body panel. In other variants, the elastic waist feature may be an integral waist portion of the front body panel and/or rear body panel of the absorbent article.

### BRIEF DESCRIPTION OF THE DRAWINGS

The absorbent articles and the method for their production as disclosed herein will be further explained hereinafter with reference to the appended drawings wherein:

FIG. 1 shows a perspective view of a variant of an absorbent pant-type article;

FIG. 2a shows a planar view of the stretched-out absorbent pant-type article in FIG. 1 with an absorbent assembly and with open side seams, as seen from an inner surface of the pant-type article;

FIG. 2b shows a schematic cross-sectional view along the longitudinal direction L of a first variant of a combined panel 4 forming the rear panel 4 of the absorbent pant-type article in FIG. 1:

FIG. 2c shows a schematic cross-sectional view along the longitudinal direction L of the front panel 3 of the absorbent pant-type article in FIG. 1;

FIG. 2d shows a schematic cross-sectional view along the longitudinal direction L of a second variant of a combined panel 4 suitable for forming the rear panel 4 of the absorbent pant-type article in FIG. 1;

FIG. 2e shows a schematic cross-sectional view along the longitudinal direction L of a third variant of a combined panel 4 suitable for forming the rear panel 4 of the absorbent pant-type article in FIG. 1:

FIG. 2f shows a schematic cross-sectional view along the longitudinal direction L of a fourth variant of a combined panel 4 suitable for forming the rear panel 4 of a variant of the absorbent pant-type article in FIG. 1.

FIGS. 3a-3d are schematic views for illustrating a variant of a method for producing a combined laminate material web;

FIG. 4 shows a planar view of a variant of a combined laminate material web;

FIG. 5 shows a schematic view to illustrate a method for producing a disposable pant-type article as shown in FIGS. 1 and 2 using a combined laminate material web as shown in FIG. 4;

FIG. 6a shows a planar view of a second variant of an absorbent pant-type article as illustrated generally in FIG. 1, the second variant being illustrated in a the stretched-out state with an absorbent assembly and with open side seams, as seen from an inner surface of the pant-type article;

FIG. 6b shows a schematic cross-sectional view along the longitudinal direction L of a variant of a combined panel 4 forming the rear panel 4 of the absorbent pant-type article in FIG. 6a; and

FIG. 6c shows a schematic cross-sectional view along the longitudinal direction L of a variant of the front panel 3 of the absorbent pant-type article in FIG. 6a.

### DETAILED DESCRIPTION

Different aspects of the present disclosure will be described more fully hereinafter with reference to the enclosed drawings. The embodiments disclosed herein can be realized in many different forms and should not be construed as being limited to the aspects set forth herein.

The absorbent pant-type article may be a baby diaper, a training pant, a feminine hygiene pant, swimwear, disposable underpants, etc.

The drawings are schematic and the pant-type article which is shown in the figures is a simplified article. It is to be understood that the article may contain further features, such as barrier cuffs, disposal means, etc. It is also to be understood that the waist elastic disclosed herein is optional or that any other suitable type of waist elastic may be used. The side seams may be reclosable side seams, and the pant-type article may be provided with fastener elements to provide reclosability of the side seams.

The method for producing a pant-type article as disclosed herein is described in the context of a disposable pant-type article comprising an absorbent assembly. However, it is to be understood that the method is equally applicable to the production of absorbent pant-type articles which do not comprise a pre-fabricated absorbent assembly. The method as disclosed herein is useful in the production of any type of disposable pant-type article, such as adult incontinence articles, baby diapers, training pants, swim-pants, disposable underpants, sanitary pants for feminine use, etc.

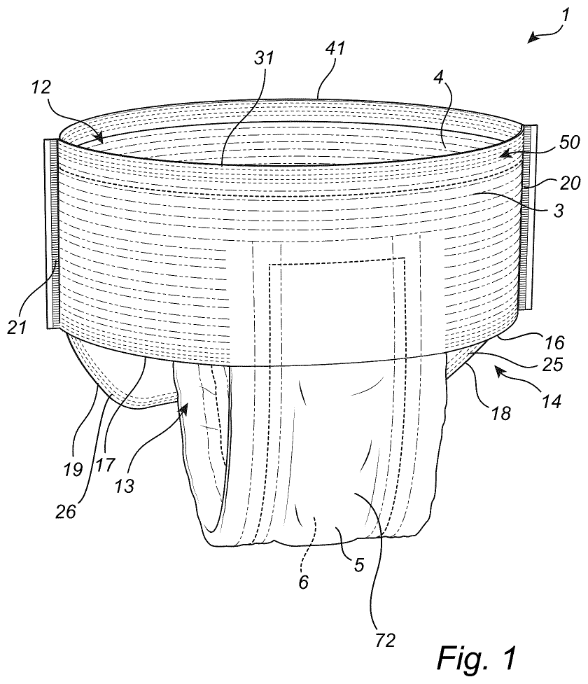

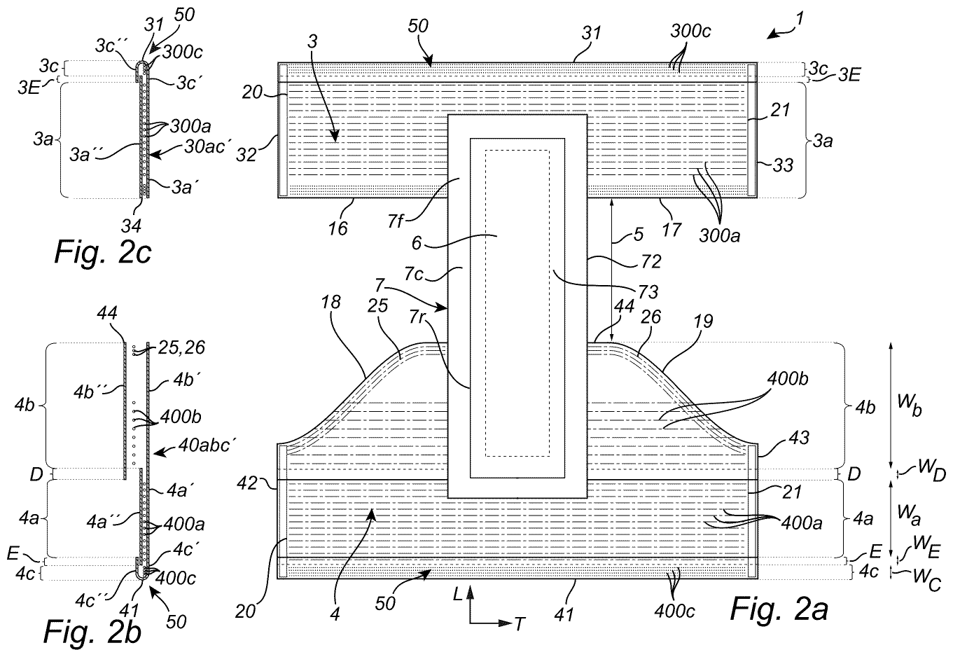

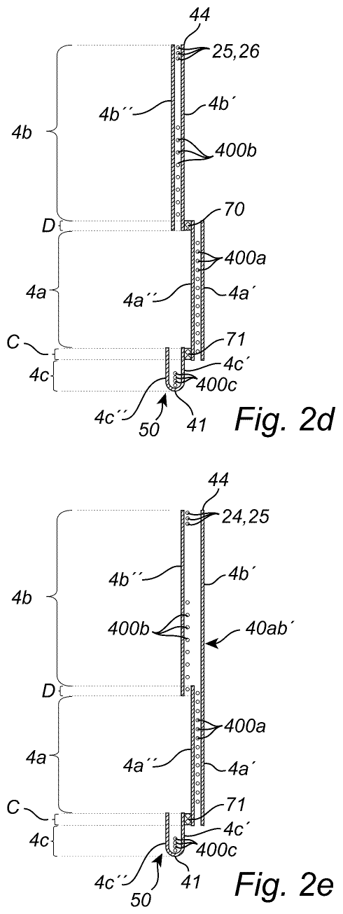

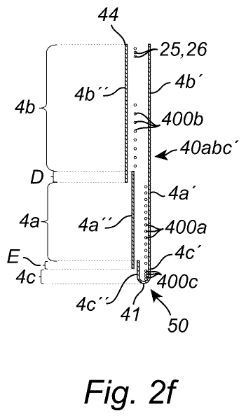

With initial reference to FIGS. 1 and 2a, there is shown a variant of a disposable absorbent pant-type article 1 in the form of a pant-type incontinence article for adult users. The pant-type article 1 is shown in FIG. 1 in an assembled condition ready for use, and in FIG. 2a in an unfolded and flat condition with the side joints 20, 21 opened up. Also in FIG. 2a, all elastic elements and components are shown in an extended state, such that the pant-type article 1 appears generally two-dimensional. FIG. 2a shows the article from an inner, wearer-facing surface of the article 1.

The pant-type article 1 has a longitudinal direction L and a transverse direction T. Any measurements of distances and dimensions are made with the pant-type article in the fully stretched-out, non-gathered condition as shown in FIG. 2a. Unless otherwise indicated, the terms “longitudinal” or “longitudinally” as used herein refer to the longitudinal direction L of the pant-type article and the terms “transverse” or “transversely” refer to the transverse direction T of the pant-type article.

The article comprises a front body panel 3 which comprises a front body panel laminate comprising elastomeric strands 300b which are elastically stretchable in the transverse direction T and a rear body panel 4, which comprises a rear body panel laminate comprising elastomeric strands 400a, 400b, which are elastically stretchable in the transverse direction T. A crotch region 5 is located between the front body panel 3 and the rear body panel 4.

The elastomeric strands 300b of the front body panel laminate are elastically stretchable in the transverse direction T and provide the overall elastic stretchability of the front body panel 3.

The elastomeric strands 400a, 400b of the rear body panel laminate provide elastic stretchability in the transverse direction T and provide the overall elastic stretchability of the rear body panel 4.

The article further comprises an absorbent core 6. The absorbent core 6 forms part of an absorbent assembly 7 which is arranged in the crotch region 5 of the pant-type article 1 and which extends in the longitudinal direction L forward in over the front body panel 3 and rearward in over the rear body panel 4. The absorbent assembly 7 bridges a gap between the front and rear body panels 3, 4. The absorbent assembly 7 which is shown in FIGS. 1 and 2 is a separately produced component, which comprises the absorbent core 6, a liquid permeable topsheet 73 and a liquid impermeable backsheet 72. The absorbent core 6 is sandwiched between the topsheet 73 and the backsheet 72.

The absorbent assembly 7 is arranged with a central part 7c of the absorbent assembly 7 located in the crotch region 5, a front end part 7f of the absorbent core overlapping with the front body panel 3 in a front overlap zone and a rear end part 7r of the absorbent assembly 7 overlapping with the rear body panel 4 in a rear overlap zone.

The absorbent core 6 which is shown in the figures has an hourglass shape. However, the disclosure is not limited to hourglass shaped cores, and it is to be understood that the absorbent core may have any useful shape, such as rectangular shape, T-shape, etc., as known in the art.

The absorbent assembly 7 is applied on the inner surface of the pant-type article 1, the inner surface being the surface which is intended to be facing the wearer when the pant-type article 1 is being worn.

The inner surface of the absorbent assembly 6 is formed by the liquid permeable topsheet 73 and may optionally include barrier cuffs not shown) extending along the side edges of the absorbent assembly 7.

The topsheet 73 and the backsheet 72 may both extend beyond the periphery of the absorbent core 6 and may be joined to each other in an edge seal extending along the periphery of the absorbent core 6 outward of a peripheral edge of the absorbent core 6. Furthermore, a peripheral edge portion of the absorbent assembly 7 may be formed by only one of the topsheet and the backsheet,

### Absorbent Core

The absorbent core 6 is disposed between the topsheet 73 and the backsheet 72 to absorb liquid, such as urine or other bodily fluids, which has passed through the topsheet 73. The topsheet 73, backsheet 72 and the absorbent core 6 may consist of any materials suitable for their purposes.

An absorbent core 6 as disclosed herein may comprise any conventional material suitable for absorbing discharged bodily wastes, such as cellulosic fluff pulp, tissue layers, highly absorbent polymers super absorbents), absorbent foam materials including hydrogel-foam material, absorbent nonwoven materials, or the like.

The absorbent core 6 may be a single-layer structure or may be a layered structure. The absorbent core 6 may comprise suitable amounts of superabsorbent material. Such superabsorbent material is well known in the field of absorbent articles and is constituted by water-swellable and water-insoluble material which is capable of absorbing large quantities of fluid upon formation of a hydrogel. The absorbent core 6 may contain superabsorbent material in the form of fibers or particles of absorbent polymer material. For example, the superabsorbent material may be surface cross-linked, partially neutralized polyacrylates. The superabsorbent material e.g., the superabsorbent fibers or particles, may be mixed with other absorbent or liquid uptake material or materials, such as cellulose fluff pulp, and/or be arranged in pockets or layers in the absorbent core 6.

The absorbent core can comprise non-absorbent components such as stiffening elements, shaping elements, binders, etc. Various types of liquid-receiving and liquid distribution elements can also be included in the core.

The absorbent core 6 may further comprise components for improving properties of the absorbent core 6, such as integrity and strength. For example, the absorbent core 6 may comprise a binder or binders, such as binder fibers. Resilient fibers, chemically stiffened fibers, etc. may be present in the absorbent core 6 to counteract wet-collapse of cellulosic fibers. Such fibers may also be useful in retaining a fluid transporting capillary network in the absorbent component so that absorbent fluid may be distributed in the absorbent component and be absorbed by superabsorbent material also in parts of the absorbent component outside the initial wetting area of the absorbent article.

The absorbent core 6 may be a three-dimensionally shaped core with different amounts of and may be provided with features such as embossings, fluid acquisition cavities and fluid distribution channels. Properties of the absorbent core such as density, amount of absorbent material and absorbent capacity may also be varied in different portions of the absorbent core.

The materials which are best suited as topsheet materials are soft and non-irritating to the skin, are readily penetrated by body fluids, and display low rewet.

### Topsheet

The liquid topsheet 73 is a wearer-facing layer and may may comprise or consist of a liquid permeable nonwoven material. Other suitable topsheet materials include tow fibres, porous foams, mesh, apertured plastic films and laminates and combinations of such materials. The topsheet may be made from thermoplastic material, such as thermoplastic synthetic fibers, film or netting. The topsheet 73 may consist of a single layer or may have a laminate structure comprising a plurality of layers, for example, two or more layers. The layers may be made of the same material, or some or all layers may be made of different materials.

### Backsheet

The liquid impermeable backsheet 72 may consist of a thin plastic film, e.g. a polyethylene or polypropylene film, a nonwoven material coated with a liquid impervious material, a hydrophobic nonwoven material which resists liquid penetration or laminates of plastic film and nonwoven. The backsheet material may be breathable to allow vapour to escape from the absorbent body, while still preventing liquids from passing through the backsheet material. The materials which may be used for the backsheet 72 include thin and flexible fluid impermeable plastic films, or fluid impermeable nonwoven materials, fluid impermeable foams and fluid impermeable laminates. The backsheet 72 may be formed by a single layer, or may be a multi-layered structure, with at least one layer being fluid impermeable.

The topsheet and backsheet may be connected to each other for example by adhesive bonding, gluing or welding by heat or ultrasonically. The topsheet and/or the backsheet may further be attached to the absorbent core by any method known in the art, such as adhesive, heat-bonding, welding, needling, etc.

### Body Panels

The front body panel 3 has a front panel waist edge 31 extending in the transverse direction T, a pair of front panel side edges 32, 33 extending in the longitudinal direction L and a front panel crotch edge extending in the transverse direction T opposite the front panel waist edge 31. On each transversal side of the absorbent assembly 7, the front panel crotch edge forms a first front leg edge 16 and a second front leg edge 17.

The rear body panel 4 has a rear panel waist edge 41 extending in the transverse direction T, a pair of rear panel side edges 42, 43 extending in the longitudinal direction L and a rear panel crotch edge extending in the transverse direction T opposite the rear panel waist edge 41. On each transversal side of the absorbent assembly 7, the rear panel crotch edge forms a first rear leg edge 18 and a second rear leg edge 19.

In the pant-type article 1 shown in the figures, the front body panel 3 has been formed from a continuous laminate material web which has been applied in a machine direction MD corresponding to the transverse direction T of the pant-type article 1. The front body panel web thereby extends in the transverse direction T across the pant-type article 1 from the second front panel side edge 32 to the first front panel side edge 33 and extends in the longitudinal direction L from the front waist edge 31 to the front crotch edge 34. The rear body panel 4 has been formed from a continuous combined laminate material web which has been applied in a machine direction MD corresponding to the transverse direction T of the pant-type article 1. The rear body panel web thereby extends in the transverse direction T across the pant-type article 1 from the first rear panel side edge 42 to the second rear panel side edge 43 and extends in the longitudinal direction L from the rear waist edge 41 to the rear crotch edge 44.

The crotch region 5 is arranged between the front body panel 3 and the rear body panel 4 and is contiguous with the front body panel 3 along the front panel crotch edge 34 and is contiguous with the rear body panel 4 along the rear panel crotch edge 44. The extension of the crotch region 5 in the longitudinal direction L is defined as the distance in the longitudinal direction between the front panel crotch edge 34 and the rear panel crotch edge 44.

The second front panel side edge 32 is intended to be joined to the second rear panel side edge 42 in a second side seam 20 and the first front panel side edge 33 is intended to be joined to the first rear panel side edge 43 in a first side seam 21 to form a pant-type article 1 having a waist opening 12, a second leg opening 13 and a first leg opening 14, as shown in FIG. 1.

The waist opening 12 is defined by the front panel waist edge 31 and the rear panel waist edge 41.

The front body panel 3 forms a pair of leg edges 16, 17, each leg edge extending transversally on a left or right side, respectively, of the absorbent assembly 7. Similarly, the rear body panel 4 forms a pair of leg edges 18, 19, each leg edge extending transversally on a left or right side, respectively, of the absorbent assembly 7. Each of the second leg opening 13 and the first leg opening 14 may be defined between a respective left or right leg edge 16, 17 of the front panel 3, a left or right leg edge 18, 19 of the rear panel 4, and a corresponding left or rear side edge of the absorbent assembly 7.

The side seams of a pant-type article as disclosed herein may be generally band-shaped joins which are formed by ultrasonic welding or thermowelding. To have sufficient strength to withstand the forces to which the pant-type article is exposed during donning of the article and to allow sufficient production tolerances when cutting off individual pant-type garments from a precursor web, the side seams commonly have a width in the order of 5 to 10 millimetres. It is also known to make side seams having a width of less than 5 mm.

It is generally desired that a soiled pant-type garment can be easily removed without having to pull the garment down over the legs of a user. Therefore, the side seams are commonly made such that they are breakable by manual force to allow a user or a caregiver to pull apart the side seams before removing a soiled pant-type garment. The pant-type article may be a reclosable article which means that the side seams can be manually opened e.g., to check if the article is soiled and needs to be changed and then closed again if it is determined that the article is in a condition allowing further use. Reclosability is commonly provided by means of fastener elements, such as hook-and-loop-type fasteners.

### Combined Panel

Thus, as explained herein, the example article 1 depicted in FIGS. 1 and 2a is a version of a disposable pant-type article 1, the article 1 having a longitudinal direction L and a transverse direction T, and defining a thickness direction perpendicular to the longitudinal direction L and the transverse direction T, wherein an inside of the article as seen in the thickness direction is a wearer facing side of the article which will be facing towards the wearer when the article is worn. The outside of the article is a garment-facing side of the article which will be facing away from the wearer when the article is worn. The article 1 comprises a front body panel 3, a rear body panel 4, and a crotch region 5 being located between the front body panel 3 and the rear body panel 4. An absorbent assembly 7 comprising an absorbent core 6 is arranged with a central part 7c of the absorbent assembly 7 being located in the crotch region 5. A first front panel side edge 32 is joined to a first rear panel side edge 42 in a first side seam 20, and a second front panel side edge 33 is joined to a second rear panel side edge 43 in a second side seam 21, and the article comprising a waist opening 12, defined by a waist edge 31 of the front panel 3 and a waist edge 41 of the rear panel 4. Further, the front body panel 3 forms a pair of leg edges 16, 17, and the rear body panel 4 forms a pair of leg edges 18, 19, the pairs of leg edges of the front panel and the rear panel defining a pair of leg openings 13, 14.

Herein, each of the front body panel 3 and the rear body panel 4 has a top side towards one of the inside and outside of the article, and a bottom side towards the other one out of the inside and outside of the article 1. “Top” and “bottom” is in this context not relating to any specific vertical or other orientation of the article 1, but merely used to define two opposite directions along the thickness dimension of a body panel 3, 4.

At least one out of the front body panel 3 and the rear body panel 4 comprises a pair of leg elastic members 25, 26 arranged adjacent the corresponding pair of leg edges.

For example, and as exemplified in the variant of FIGS. 1 and 2a, the rear body panel 4 of the absorbent article 1 may comprise a pair of leg elastic members 25, 26 arranged adjacent the pair of leg edges 18, 19 of the rear body panel 4. The leg elastic members 25, 26 may be arranged along the complete or part of the rear parts of the leg edges 18, 19 of the rear body panel 4. The leg edges 18, 19 are curved so as to form leg openings comfortably following the leg of wearer. The leg edges 18, 19 being curved implies that the leg edges 18, 19 extend in the transversal direction T and in the longitudinal direction L.

Each leg elastic member 25, 26 may comprise individual elastic elements, such as 2 to 15 individual elastic elements, 3 to 12 individual elastic element or 4 to 10 individual elastic elements. The individual elastic elements may be elastic strings, threads or bands which are applied spaced apart along the rear leg edge sections of the second and first leg edges.

The leg elastic members 25, 26 each extends at least partially in a direction other than parallel with the transversal direction T. Accordingly, the leg elastic members 25, 26 may follow the contour of the leg opening so as to allow for an improved fit of the absorbent article around the leg of the wearer.

For example, at least a part of each leg elastic member 25, 26 may form an angle of at least 20 degrees with the transversal direction T, such as at least 30 degrees with the transversal direction T. For example, at least a part of each leg elastic member 25, 26 may form an angle of from 20 degrees to 80 degrees with the transversal direction T, such as from 30 degrees to 60 degrees with the transversal direction T. For example, as in the variant of the figures, at least a part of each leg elastic member 25, 26 forms an angle of about 45 degrees with the transversal direction T.

For example, each leg elastic member 25, 26 may form an angle of at least 20 degrees with the transversal direction T over at least 10% of the total width as seen in the transversal direction T of the combined panel 4. For example, each leg elastic member may form an angle as set out in the above over at least 10%, such as at least 20% of the total width of the combined panel 4.

For example, as may be seen in the example of FIG. 2a, the leg elastic members 25, 26 may substantially follow a curved shape of the leg edges 18, 19.

As mentioned in the introduction of this application, it has been found to be challenging to apply leg elastic members 25, 26 which at least partially extends in a direction other than parallel with the transversal direction T using techniques involving welded bond patterns.

Thus, the present inventors have proposed a combined panel 4 which comprises such leg elastic members 25, 26, and which comprises a first panel portion 4a and a second panel portion 4b, as seen along the longitudinal direction L. In the disclosed combined panel 4, the first panel portion 4a and the second panel portion 4b are bonded using different bonding techniques, as will be further explained in the below.

Herein, the term “combined panel” is used to denote a panel comprising leg elastic members 25, 26 which at least partially extends in a direction other than parallel with the transversal direction T, and which comprises a first panel portion 4a and a second panel portion 4b being bonded using welding techniques and adhesive techniques, respectively, as defined in the appended claims. Variants and features of the combined panel will be further described in the below.

In the following, the description will be made in relation to a variant of an absorbent article 1 as depicted in the Figs, wherein the rear body panel 4 is a combined panel comprising leg elastic members 25, 26, However, it should be understood that the below description of a combined panel may equally be applied to a front body panel 3 being a combined panel comprising leg elastic members.

As exemplified in FIG. 2a and in FIG. 2b (showing a cross section in a direction along the longitudinal axis L of the combined panel 4 in FIG. 2a), the first panel portion 4a comprises a first bottom layer 4a′ and a first top layer 4a″ being superimposed such that, in the first panel portion 4a, the first bottom layer 4a′ forms a bottom side surface and the first top layer 4a″ forms a top side surface of the combined panel 4 (see FIG. 2b).

Similarly, the second panel portion 4b comprises a second bottom layer 4b′ and a second top layer 4b″ being superimposed such that, in the second panel portion 4b, the second bottom layer 4b′ forms a bottom side surface and the second top layer 4b″ forms a top side surface of the combined panel 4.

The bottom side surface of the combined panel is the outermost surface of the panel as seen towards the bottom side of the panel, and the top side surface of the combined panel is the outermost surface of the panel as seen towards the top side of the panel. Accordingly, in each out of the first and second panel portion 4a, 4b, the respective bottom layer 4a′, 4b′ and top layer 4a″, 4b″ pair forms the bottom side surface and the top side surface of the combined panel 4, i.e. forms the inside surface and the outside surface of the combined panel.

As will be further described in the below, the pairs of the first/second bottom layer 4a′, 4b′ and the first/second top layer 4a″, 4b″ are sandwiching elastomeric strands and/or elastic members to form the elastic rear panel 4. By virtue of the top layer forming a top side surface and the bottom layer forming a bottom side surface of the combined panel 4 in the first panel portion 4a and in the second panel portion 4b, the combined panel 4 may be formed to be relatively thin and without using unnecessary amounts of material.

As illustrated in FIG. 2a and FIG. 2b, the first panel portion 4a and the second panel portion 4b are connected by a connection region D.

The connection region D joins the first panel portion 4a and the second panel portion 4b. Thus, in the connection region, the top and/or bottom layer of the first panel portion 4a may for example be arranged so as to overlap the top and/or bottom layer of the second panel portion 4b. Different variants of the connection region D will be more described in the below.

As illustrated in FIG. 2a and FIG. 2b, the first panel portion 4a extends from the connection regionD and in a longitudinal direction towards the waist edge 41 of the combined panel 4, and the second panel portion 4b extends from the connection region D and in a longitudinal direction towards the leg edges 18, 19 of the combined panel 4. For example, the second panel portion 4b may extend longitudinally from the connection region D and all the way to the leg edges 18, 19 of the combined panel 4.

The second panel portion 4b comprises the leg elastic members 25, 26. In contrast, the first panel portion 4a is free from leg elastic members 25, 26.

However, the first panel portion 4a comprises a first set of elastomeric strands 400a being sandwiched between the first bottom layer 4a′ and the first top layer 4a″. The elastomeric strands 400a are aligned in parallel with each other and substantially with the transversal direction T, and the elastomeric strands 400a are spaced apart from each other in the longitudinal direction. As such, the first set of elastomeric strands 400a imparts elasticity in the transversal direction T to the first panel portion 4a of the combined panel 4.

In the first panel portion 4a, the first bottom layer 4a′ is bonded to the first top layer 4a″ by a welded bond pattern for attaching the first set of elastomeric strands 400a between the first bottom layer 4a′ and the first top layer 4a″. Since the first panel portion 4a is free from leg elastic members 25, 26, i.e., includes only elastic strands 400a which are substantially aligned with the transversal direction T, numerous techniques for bonding involving a welded bond pattern are available, which will be further described in the below. Thus, the first panel portion 4a may bonded by a welded bond pattern and may be free from adhesive.

The second panel portion 4b comprises the previously mentioned second bottom layer 4b′, the second top layer 4b″, and the leg elastic members 25, 26. In some variants, such as the one illustrated in FIGS. 2a and 2b, the second panel portion 4b further comprises a second set of elastomeric strands 400b. The leg elastic members 25, 26 and the second set of elastomeric strands 400b are sandwiched between the second bottom layer 4b′ and the second top layer 4b″, the elastomeric strands 400b being aligned in parallel with each other and substantially with the transversal direction T, and the elastomeric strands 400b being spaced apart from each other in the longitudinal direction L.

Thus, the second set of elastomeric strands 400b impart elasticity in a direction parallel to the transversal direction T to the second panel portion 4b of the combined panel 4.

The second panel portion 4b comprises the leg elastic members 25, 26 arranged adjacent the leg edges 18, 19. The leg elastic members 25, 26 are sandwiched between the second top layer 4b″ and the second bottom layer 4b′, just as the optional elastomeric strands 400b.

The second panel portion 4b is bonded by adhesive bonds, wherein the adhesive bonds are applied to one or more out of the second top layer 4b″, the second bottom layer 4b′, the second set of elastomeric strands 400b, and the leg elastic members 25, 26. To this end, prior known adhesive techniques may be used.

In view of the above, in the combined panel 4, the first panel portion 4a is bonded by means of a welded bond pattern, whereas the second panel portion 4b is joined by means of adhesive. As such, adhesive bonding is used only where required, i.e., to accomplish the attachment of the leg elastics 25, 26. Since the leg elastics 25, 26 form a curved shape, other bonding methods than adhesive bonding methods may be less suitable in the portion 4b of the combined panel 4. By forming the combined panel 4 of a first portion 4a and a second portion 4b being joined along a transversal connection region D, different bonding methods may be used in the first portion 4a and in the second portion 4b, without causing unnecessary consumption of material.

Optionally, and as in the illustrated variant, each leg elastic member 25, 27 crosses at least one elastic strand out of the second set of elastomeric strands 400b.

In the first panel portion 4a and in the second panel portion 4b, respectively, the top layer 4a″, 4b″ forms a top side surface and the bottom layer 4a′, 4b′ forms a bottom side surface of the combined panel 4. That no additional layers of web material are present in the combined panel 4 over the first panel portion 4a and the second panel portion 4b may be advantageous in terms of breathability, comfort, sustainability, and economics.

The connection region D serves the purpose of connecting the first and second panel portions only and may thus be made so as to have a length Wd being as short as possible. In the connection region D, the combined panel 4 may comprise more than two layers, for example three or more layers, to provide the desired connection between the first and second panel portions.

For example, the connection region D may have a longitudinal length Wd being no more than 10% of a total longitudinal length of the combined panel, such as no more than 7% or no more than 5% of a total longitudinal length W of the combined panel. In the illustrated example, the connection region D may have a longitudinal length Wd corresponding to from 4 to 5% of the longitudinal length W of the combined panel 4.

The first bottom layer 4a′, the first top layer 4a″, the second bottom layer 4b′ and the second top layer 4b″ may be selected so as to provide the combined panel 4 with a relatively uniform appearance, despite the division into a first and a second panel portion 4a, 4b, and the presence of the connection region D.

In some variants, such as in the examples of FIG. 2b, the first bottom layer 4a′ and the second bottom layer 4b′ are both formed by the same, single sheet of material 40abc′, extending longitudinally over the connection region D.

The first top layer 4a″ and the second top layer 4b″ may advantageously be formed by separate sheets of sheet material, as in the variant of FIG. 2b. As such, in production, each of the separate sheets may be bonded to form the first portion 4a or the second portion 4b, respectively, with the respective welding or adhesive bonding process.

Still, the second top layer 4b″ and the first top layer 4a″ may be formed by sheets of identical sheet material. For example, the second top layer 4b″ and the first top layer 4a″ may be formed by separate sheets being slitted from the same original (broader) sheet material.

Variants where, as in FIG. 2b, the first bottom layer 4a′ and the second bottom layer 4b′ are formed by a single sheet of material 40ab′, 40abc′ extending over the connection region D have the further advantage that the connection region D may comprise relatively few layers of sheet material. For example, when as in FIG. 2b the sheet material of the first top layer 4a″ and the sheet material of the second top layer 4b″ are arranged so as to overlap in the connection region D, the connection region still comprises no more than three layers of sheet material.

Thus, the combined panel 4 may comprise a first panel portion 4a and a second panel portion 4b connected by the connection region D as set out in the above, wherein the first panel portion 4a and the second panel portion 4b each comprises no more than two layers of sheet material, and the connection region D comprises no more than three layers of sheet material. Accordingly, the connection region D may be made with a lesser impact on the breathability and comfort of the combined panel 4.

The first panel portion 4a has a longitudinal length Wa which may be significant portion of the total length W of the combined panel. For example, the longitudinal length Wa may be at least 20% of a total longitudinal length of the combined panel, such as at least 25%, or at least 30%. In the illustrated example, the longitudinal length Wa of the first panel portion 4a is from 30% to 40% of the longitudinal length W of the combined panel 4. However, it will be understood that this proportion may be increased or decreased for example depending on the size and model of the absorbent article.

The second panel portion 4b has a second longitudinal length Wb which may be significant portion of the total length W of the combined panel 4. For example, the longitudinal length Wb of the second panel portion 4b may be at least 20%, such as at least 25% or at least 30% off a total longitudinal length of the combined panel 4. For example, the second panel portion may have a longitudinal length being less than 70% of the total longitudinal length of the combined panel 4. For example, the second panel portion may have a longitudinal length being from 25% to 70% of the total longitudinal length of the combined panel 4.

In the absorbent article 1, the absorbent assembly 7 may be arranged with a central part 7c of the absorbent assembly 7 being located in the crotch region 5 and with a front end part 7f of the absorbent assembly 7 overlapping with the front body panel 3 in a front overlap area and with a rear end part 7r of the absorbent assembly 7 overlapping with the rear body panel 4 in a rear overlap area.

As exemplified in FIG. 2a, the absorbent assembly 7 may be arranged so as to overlap a part of the connection region D. In other words, the rear end part 7r of the absorbent assembly 7 extends longitudinally beyond the transversal connection region D in a direction towards the rear waist edge 41 of the article 1.

Further, the pant-type article 1 as disclosed herein may have an elastic waist feature 50 arranged along the waist opening 12 (See FIG. 1.) An elastic waist feature 50 may include one or more elastic elements extending parallel with the front panel waist edge 31 and the rear panel waist edge 41. The elastic waist element or elements may be incorporated in the combined 4 or may be applied as a separate waistband which is attached to the front panel waist edge 31 and the rear panel waist edge 41 or which is attached to the front and/or to the rear body panel 3, 4 with the waistband completely overlapping with the corresponding body panel 3, 4 and sharing a common waist edge. The pant-type article 1 which is shown in the Figures has an elastic waist feature 50 which extends around the full circumference of the waist opening 12. However, an elastic waist feature may be arranged only along a part of the waist opening, such as only along the rear panel waist edge, only along the front panel waist edge or along a part of one or both of the front panel waist edge and the rear panel waist edge, which part has a length which is less than the full length of the corresponding panel waist edge.

For example, to form an elastic waist feature 50, the combined panel 4 may comprise a waist panel portion 4c as exemplified in FIG. 2b.

In the waist panel portion 4c, a waist bottom layer 4c′ and a waist top layer 4c″ are superimposed such that the waist bottom layer 4c′ forms a bottom side surface and the waist top layer 4c″ forms a top side surface of the combined panel 4.

The waist panel portion 4c further comprises a third set of elastomeric strands 400c being sandwiched between the waist bottom layer 4c′ and the waist top layer 4c″, the elastomeric strands 400c being aligned substantially in parallel with each other and with the transversal direction T, and the elastomeric strands 400c being spaced apart from each other in the longitudinal direction L.

The waist panel portion 4c is joined to the first panel portion 4a by a waist connection region E. As such, the first panel portion 4a extends longitudinally from the connection region D to the waist connection region E. The waist panel portion 4c extends from the waist connection region E to the waist edge 41 of the combined panel 4.

Similar to the connection region D, it is preferred that the waist connection region E has a relatively short longitudinal length WE, for example no more than 10% or no more than 5% of the total longitudinal length W of the combined panel 4.

Numerous options for forming a waist panel portion 4c on a front or rear body panel are known and may be combined with the present disclosure. For example, the different variants described in WO 2018/09771 A1 may be used on the front or rear body panels as described herein. For example, the different variants as described in EP 3 788 997 A1 may be used on the front or rear body panels as described herein.

For example, as depicted in the variant of FIG. 2b, the waist bottom layer 4c′ and the waist top layer 4c″ may be formed from a continuous material sheet being folded upon itself along the waist edge 41. Since the waist panel portion 4c is free from elastic members extending in directions other than substantially parallel to the transversal direction T, the waist panel portion 4c may advantageously be bonded by welded bond pattern bonding the waist bottom layer 4c′ to the waist top layer 4c″ so as to attach the third set of elastic strands 400c therebetween. Again, this implies that adhesives are used in the combined panel 4 only when required by the need for securely bonding leg elastic members, and that adhesives are otherwise avoided.

In some variants, such as exemplified in FIG. 2d, the waist bottom layer 4c′, the waist top layer 4c″, the first bottom layer 4a′ and the second bottom layer 4b′ may be formed by a single continuous material sheet 40abc′. As such, a uniform appearance of the bottom side of the panel may be achieved, being particularly desired when the bottom direction is the outward direction of the article 1. Moreover, advantages may be obtained in manufacturing as will be described in the below. To form the waist top layer 4c″ from the continuous material sheet 40 abc′, the sheet 40abc′ is folded along a folded edge thus forming the waist edge 41 of the combined panel 4.

In the variant of FIG. 2b, the first top layer 4a″ and the second top layer 4b″ are formed by separate material sheets which overlap in the connection region D. Similarly, the material sheets of the first top layer 4a″ and the waist top layer 4c″ overlap in the longitudinal direction L in the waist connection region E.

FIGS. 2d, 2e, and 2f illustrate different examples of possible cross-sections through a variants of a combined panel 4 as generally illustrated of FIG. 2a, as seen along the longitudinal directionL, thus illustrating different variants of connection regions D and waist features 50.

In the variant of FIG. 2d, the first bottom layer 4a′ and the second bottom layer 4b′ are formed by separate material sheets. Moreover, the first top layer 4a″ and the second top layer 4b″ are formed by separate material sheets. The connection region D comprises a joint 70 connecting the first panel portion 4a and the second panel portion 4b. As may be seen in FIG. 2d, the joint 70 may for example be applied such that the first top layer 4a″ and the first bottom layer 4a′ overlap with the second top layer 4b″ and the second bottom layer 4b′ in the connection region D. The joint 70 may be any suitable joint, such as an adhesive joint or a welded joint.

Further, the combined panel 4 in the variant of FIG. 2d comprises a waist feature 50 in the form of a waist panel portion 4c. In the waist panel portion 4c, a waist bottom layer 4c′ and a waist top layer 4c″ are superimposed such that the waist bottom layer 4c′ forms a bottom side surface and the waist top layer 4c″ forms a top side surface of the combined panel 4.

The waist panel portion 4c further comprises a third set of elastomeric strands 400c being sandwiched between the waist bottom layer 4c′ and the waist top layer 4c″, the elastomeric strands 400c being aligned substantially in parallel with each other and with the transversal direction T, and the elastomeric strands 400c being spaced apart from each other in the longitudinal direction L.

For example, as depicted in the variant of FIG. 2d, the waist bottom layer 4c′ and the waist top layer 4c″ may be formed from a continuous material sheet being folded upon itself along the waist edge 41.

The waist panel portion 4c is joined to the first panel portion 4a by a waist connection region E.

Since the waist panel portion 4c is free from elastic members extending in directions other than substantially parallel to the transversal direction T, the waist panel portion 4c may advantageously be bonded by welded bond pattern bonding the waist bottom layer 4c′ to the waist top layer 4c″ so as to attach the third set of elastic strands 400c therebetween. Again, this implies that adhesives are used in the combined panel 4 only when required by the need for securely bonding leg elastic members, and that adhesives are otherwise avoided.

The waist connection region E may be made in any suitable manner. For example, as in the variant illustrated in FIG. 2d, the waist connection regionE may comprise a joint 71, which may be a joint such as described in relation to the joint 70 of the connection region D in the above.

For example, and as illustrated in FIG. 2d, the waist bottom layer 4c′ and the waist top layer 4c″ may be arranged so as to overlap with the first bottom layer 4a′ and the first top layer 4a″ in the waist connection region E.

As mentioned in the above, similar to the connection region D, it is preferred that the waist connection region E has a relatively short longitudinal length WE, for example no more than 10% or no more than 5% of the total longitudinal length W of the combined panel 4.

In the example of FIG. 2d, each one out of the second panel portion 4b, the first panel portion 4a, and the optional waist panel portion 4c may be produced separately and then joined via the joints 70, 71 to form the combined panel 4. As such, it may be advantageous to form a second laminate web 4b, a first laminate web 4a, and optionally a waist laminate web, and to join the webs via joints 70, 31 so as to form a combined laminate web having a cross-section as illustrated in FIG. 2d, and which may be cut into rear body panels 4 during manufacture of the pant-type articles 1.

FIG. 2e illustrates another variant of a combined panel 4 as seen in a cross-section along the longitudinal direction of FIG. 2a.

In this variant, the second bottom layer 4b′ of the second panel portion 4b and the first bottom layer 4a′ of the first panel portion 4a are formed by a single continuous material sheet 40ab′, extending over the connection region D. As seen in FIG. 2e, in such variants, a combined bottom layer 40ab′ constitutes the second bottom layer 4b′ in the second portion 4b of the combined panel 4 and the first bottom layer 4a′ in the first portion 4a of the combined panel 4. This gives the combined panel 4 a uniform appearance when seen from the bottom layer side. For example, as in the illustrated variant, the bottom direction may be the outer direction of the article 1, in which case the uniform appearance of the combined bottom layer 40ab′ will be visible from the outside of the article 1. Thus, any joints or overlap layers may be avoided on the outside of the article 1. Further, as will be explained in the below, the second bottom layer 4b′ and the first bottom layer 4a′ being formed by a single continuous material sheet may provide for advantageous manufacturing.

As illustrated in FIG. 2e, the first top layer 4a″ of the first panel portion 4a, and the second top layer 4b″ of the second panel portion 4b are formed by separate material sheets which are arranged with an overlap the longitudinal direction L in the connection region D.

In the example of FIG. 2e, the combined panel 4 further comprises a waist feature 50. The waist feature 50 may be any of the waist features as described in the above in relation to the example of FIG. 2b or FIG. 2d. For example, as in the variant of FIG. 2e, the waist feature 50 may comprises a waist panel portion 4c being joined to the first panel portion 4a via a joint 71, similar to what has already been described in relation to FIG. 2d.

Other variants than those depicted in the Figures are or course possible. For example, the waist bottom layer 4c′ and the first bottom layer 4a′ may be formed by a single continuous material sheet as described in relation to FIG. 2b, even in variants where the second panel portion 4b and the first panel portion 4a do not share a continuous material sheet, but rather are joined by a joint 70 as described in relation to FIG. 2d.

When the connection region D or the waist connection region E involves an overlap such as an overlap between separate sheet materials of the top layers only, the overlap could be made in any direction.

For example, FIG. 2f illustrates a variant largely similar to the variant of FIG. 2b, apart from that in this variant, in the waist connection region E, the first top layer 4a″ is arranged to the top side of the waist top layer 4c″ (whereas in FIG. 2b, the first top layer 4a″ is arranged to the bottom side of the waist top layer 4c″). The relative arrangement of the layers in the waist connection region E may reflect the order in which the waist portion 4c and the first panel portion 4b, respectively, was formed when manufacturing the combined panel 4.

Different relative arrangements of the first top layer 4a″ and the second top layer 4b″ are also possible in the connection region D. However, in the connection region D it may be preferable if the second top layer 4b″ is arranged to the top side of the first top layer 4a″ since this implies that the welded pattern of the first panel portion 4a may be produced prior to the adhesive of the second panel portion 4b, using a continuous material 4ab′ to form the first bottom layer 4a′ and the second bottom layer 4b′. Accordingly, any risks of having adhesive in the welding equipment may be avoided.

Moreover, the connection region D and/or the waist connection region E could be close to infinitely small. For example, in a variant where a first top layer 4a″ is arranged edge to edge with a second top layer 4b″ on a continuous material 4ab′ forming the first bottom layer 4a′ and the second bottom layer 4b′, the connection region D would comprise only the line between the edges of the first bottom layer 4a′ and the second bottom layer 4b′.

The first bottom layer 4a′, the first top layer 4a″, the second bottom layer 4b′ and the second top layer 4b″ may be selected so as to provide the combined panel 4 with a relatively uniform appearance, despite the division into a first and a second panel portion 4a, 4b, and the presence of the connection region D.

In some variants, such as in the examples of FIG. 2b, FIG. 2e, and FIG. 2f the first bottom layer 4a′ and the second bottom layer 4b′ may be formed by a single sheet of material 40ab′, 40abc′ extending over the connection region D.

In other variants, such as in the example of FIG. 2d, the second bottom layer 4b′ and the first bottom layer 4a′ may be formed by different sheets of material 4a′, 4b′. Although giving a less uniform appearance than the versions of FIG. 2b, FIG. 2e and FIG. 2f, till, in this case, the different sheets may be made of identical sheet material. For example, the first bottom layer 4a′ and the second bottom layer 4b′ may be formed by separate sheets being slitted from the same original (broader) sheet material.

The first top layer 4a″ and the second top layer 4b″ may advantageously be formed by separate sheets of sheet material, as in the variants of the Figures. As such, in production, each of the separate sheets may be bonded to form the first portion 4a or the second portion 4b, respectively, with the respective welding or adhesive bonding process.

Still, the second top layer 4b″ and the first top layer 4a″ may be formed by sheets of identical sheet material. For example, the second top layer 4b″ and the first top layer 4a″ may be formed by separate sheets being slitted from the same original (broader) sheet material.

Variants where, as in FIG. 2b, FIG. 2e or FIG. 2f, the first bottom layer 4a′ and the second bottom layer 4b′ are formed by a single sheet of material 40ab′, 40abc′ extending over the connection region D have the further advantage that the connection region D may comprise three layers of sheet material only.

Although in the illustrated variant, the rear panel 4 only comprises leg elastic members and is a combined panel, thus comprising a second panel portion 4b and a rear panel portion 4a as described in the above, it will be understood that other variants are possible. For example, also the front panel 3 may comprise leg elastic members and/or may be a combined panel comprising a first panel portion and a second panel portion as described in the above.

In some variants however, such as in the variant of FIG. 2a, the front body panel 3 of the absorbent article is free from leg elastic members 25, 26. As seen in FIG. 2a and FIG. 2c (FIG. 2c showing a cross-section in the longitudinal direction L of the front body panel 3 in FIG. 2a, the front body panel 3 comprises a main bottom layer 3a′ and a main top layer 3a″ and a set of elastomeric strands 300b being sandwiched between the main bottom layer 3a′ and the main top layer 3a″, the elastomeric strands 300a being aligned in parallel with each other and substantially with the transversal direction T, and the elastomeric strands 300a being spaced apart from each other in the longitudinal direction L.

In the main portion 3a, the main bottom layer 3a′ is bonded to the main top layer 3a″ by a welded bond pattern for attaching the set of elastomeric strands 300a.

In the main portion 3a, the main bottom layer 3a′ forms a bottom side surface and the main top layer 3a″ forms a top side surface of the front body panel 3.

The front body panel 3 may be provided with a waist feature 50, similar to what has been described in the above. In the variant of FIG. 2c, the front body panel 3 comprises a waist panel portion 3c comprising a top waist panel layer 3c″ and a bottom waist panel layer 3c′, where the bottom waist panel layer 3c′ is formed by a combined bottom panel sheet 30ac′, which also forms the main bottom layer 3a′, similar to the waist feature 50 as described in relation to FIGS. 2b and 2c.

Preferably, the waist panel portion 3c of the front body panel 3 may be bonded by a welded bond pattern as described in the above in relation to the combined panel 4.

The front body panel 3 being free from leg elastic members 25, 26 extending in a direction other than parallel with the transversal direction T implies that the front body panel 3 as a whole may suitably be bonded by a welded bond pattern. Thus, the front body panel 3 may be free from adhesive.

In some variants, as depicted in FIGS. 2a and 2c, the set of elastomeric strands 300a in the main portion 4a may be divided into a first subset and a second subset, where the first subset of elastomeric strands differs from the second subset of elastomeric strands as regards longitudinal spacing between the elastic strands, and/or features of the elastic strands such as diameter of the elastic strands. For example, the first subset of elastomeric strands may be applied over a majority of the longitudinal extension of the main panel portion 3a, whereas the second subset of elastomeric strands is applied adjacent the leg edges 16, 17 of the front panel portion 3. As such, the second subset of elastomeric strands may be provided to ensure a good fit of the front panel 3 at the leg openings 13, 14 of the absorbent article 1. For example, the second subset of elastomeric strands may have a longitudinal spacing between each strand being less than the longitudinal spacing of the first subset of elastomeric strands. The second subset of elastomeric strands may comprise a relatively low number of elastomeric strands, such as from 1 to 5 strands, or, as in the example, from 2 to 4 strands.

In other variants, the front panel 3 may be provided with a leg panel portion which may be formed in the same manner as previously described for the waist panel portion, to ensure a good fit of the front panel 3 at the leg openings 13, 14 of the absorbent article. Thus, a leg panel portion may be connected to the main panel portion 3a via a leg connection portion, similar to the waist connection portion C described in the above.

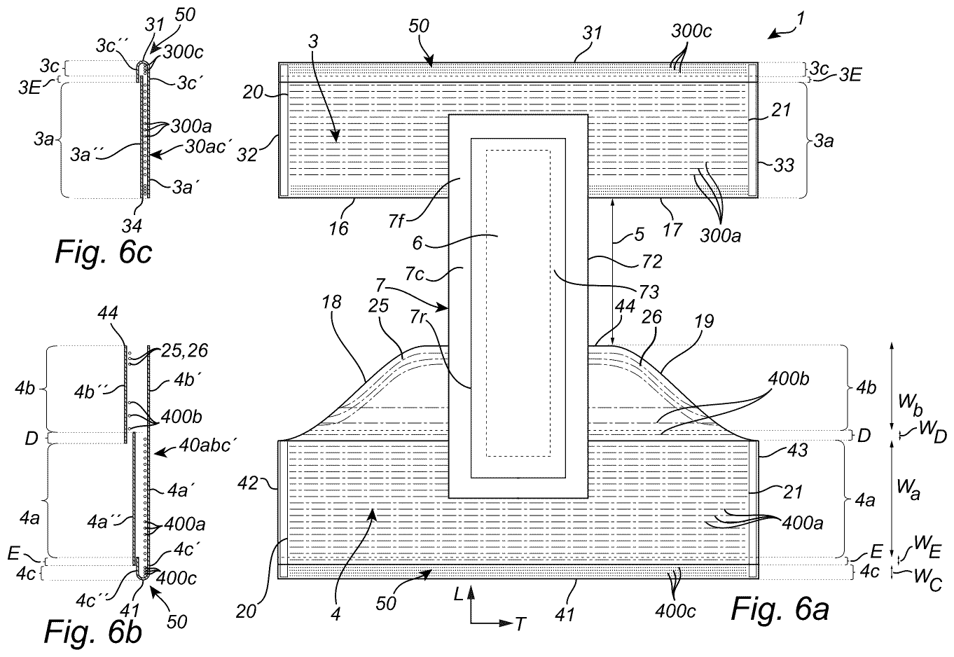

FIGS. 6a, 6b, and 6c illustrate yet another variant of an absorbent article 1 with another variant of a combined panel 4. As seen in FIG. 6a, and in FIG. 6c, apart from the rear body panel 4 being another variant of a combined body panel 4, the absorbent article 1 is similar to the article 1 illustrated in FIG. 2a with the front panel 3 illustrated in FIG. 2c, and the description made in relation to the variants of FIGS. 2a and 2c apply also to the variant illustrated in FIGS. 6a and 6b. The rear body panel 4 of the variant in FIG. 6a is shown in cross-section in FIG. 6c. As seen in FIG. 6b, this variant of a combined body panel 4 differs from the one e.g. in FIG. 2b in that each leg elastic member 25, 26 terminate at the respective leg edge 18, 19. Thus, each leg elastic member 25, 26 does not extend all the way to the first rear panel side edge 42 or the second rear panel side edge 43, respectively. Accordingly, each elastic member 25, 26 does not extend to the first side seam 20 or the second side seam 21, respectively. This provides for a possibility of making the second panel portion 4b relatively shorter as seen in the longitudinal direction L, and thus to make the first panel portion 4a relatively longer. A relatively longer first panel portion 4a may be preferred since it is generally desired to increase the use of welding bonding techniques so as to diminish use of adhesive bonding techniques.

In the variant of FIGS. 2a and 2b, the second panel portion 4b comprises a second set of elastic strands 400b including a relatively low number of strands 400b. However, the second set of elastic strands 400b may optionally be completely omitted such that the second panel portion 4b comprises no elastic elements but the leg elastic members 25, 26.

### Combined Laminate Material Web

In in-line manufacturing of absorbent articles 1, the front body panel 3 and the rear body panel 4 of the absorbent article 1 as depicted in FIG. 2a may be achieved by cutting the front body panel and the rear body panel from a respective front body panel web or a rear body panel web.

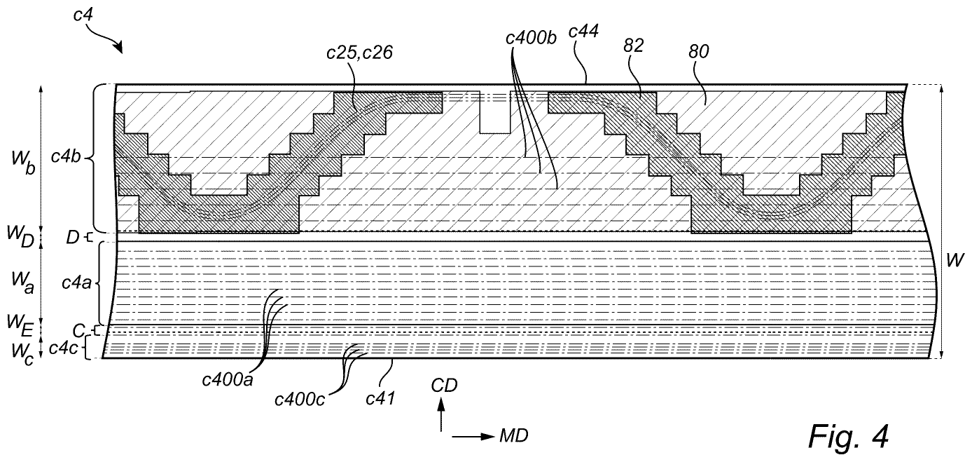

FIG. 4 schematically illustrates a variant a combined laminate material web c4 which may be cut for forming combined body panels 4 as described in the above. The illustrated combined laminate material web c4 is for forming combined panels 4 in accordance with the variant of FIGS. 2a and 2b. However, it will be understood that other variants of combined laminate material webs may be formed, such as variants corresponding to the other variants of combined panels illustrated herein.

Thus, FIG. 4 illustrates a variant of a combined laminate material web c4 for a disposable hygienic article, the combined laminate material web being continuous as seen in a machine direction MD and having a width W between two side edges 500, 501 in a cross-direction CD, perpendicular to the machine direction MD, the combined laminate material web defining a top direction being perpendicular to the machine direction and to the cross-direction CD, and a bottom direction opposing the top direction. The combined laminate material web c4 comprises a first web portion c4a and a second web portion c4b, the first web portion c4a and the second web portion c4b being connected by a connection region D extending along the machine direction MD, such that the first web portion C4a extends from the connection region D and in the cross-direction towards the first side edge c41 of the combined laminate material web, and the second web portion c4b extends from the connection region D and in the cross-direction towards the second side edge c44 of the combined laminate material web.

The first web portion c4a comprises a first bottom layer 4a′ and a first top layer 4a″ being superimposed such that, in the first web portion 4a, the first bottom layer 4a′ forms a bottom side surface and the first top layer 4a″ forms a top side surface of the combined laminate material web c4.

Similarly, the second web portion 4b comprises a second bottom layer 4b′ and a second top layer 4b″ being superimposed such that, in the second web portion 4b, the second bottom layer 4b′ forms a bottom side surface and the second top layer 4b″ forms a top side surface of the combined laminate material web c4.

The first web portion c4a further comprises a first set of elastomeric strands c400a being sandwiched between the first bottom layer 4a′ and the first top layer 4a″, the elastomeric strands c400a being aligned in parallel with each other and substantially with the machine direction MD, and the elastomeric strands being spaced apart from each other in the cross-direction CD, and the first web portion c4a is bonded by the first bottom layer 4a′ being bonded to the first top layer 4a″ by a welded bond pattern for attaching the first set of elastomeric strands 400a between the first bottom layer 4a′ and the first top layer 4a″.

The second web portion c4b comprises a second set of elastomeric strands 400b being sandwiched between the second bottom layer 4b′ and the second top layer 4a′, the elastomeric strands 400b being substantially aligned in parallel with each other and with the machine direction MD, and the elastomeric strands 400b being spaced apart from each other in the cross-direction CD. The second web portion 4b further comprising additional elastic members c25, c26 extending at least partially in a direction other than parallel with machine direction MD, wherein the additional elastic members 25, 26 are sandwiched between the second bottom layer 4b′ and the second top layer 4b″, and the second web portion 4b is bonded by the second bottom layer 4b′ being bonded to the second top layer 4b″ by adhesive bonds, for attaching the second set of elastomeric strands 400b and the additional elastic members 25, 26 between the second bottom layer 4b′ and the second top layer 4b″.

The additional elastic members c25, c26 of the second web portion c4b are to form the leg elastic members 25, 26 in the absorbent articles 1 including combined panels 4 cut from the combined laminate web c4. To that end, the additional elastic members c25, c26b may, as in the illustrated variant, run continuously and in a meandering pattern over the second web portion 4b of the combined laminate web c4.

As will be described in more detail in the below in relation to a method for forming an absorbent article 1, the manufacturing of an absorbent article 1 will involve cutting portions of the combined laminate web c4 away so as to form the leg edges 18, 19 of the combined panels 4, and cutting the combined laminate web c4 into separate rear panels 4 by cuts along the cross-direction along the dotted lines in FIG. 4. Thus, in the combined panels 4, the additional elastic members c25, c26 will be positioned adjacent the leg edges 18, 19. Finally, the additional elastic members c25, c26 which may run continuously as seen in the machine direction MD of the combined laminate web c4 may be centrally cut or otherwise de-elastified in a central section of each rear panel 4, so as to form a first elastic members 25 along a first leg edge 18, and a second elastic member 26 along a second leg edge 19 in the final rear panel 4. Such cutting or de-elastification of the additional elastic members c25, c26 in a central section of each rear panel 4 may be performed prior to cutting the combined laminate web c4 into separate rear panels 4.

As explained in the above in relation to the article 1 comprising the rear panel 4, a combined laminate web 4 with a first web portion 4a and a second web portion 4b as described in the above may be constructed in different manners, such as exemplified in relation to FIGS. 2d to 2b, or 2f, or in relation to FIGS. 6a and 6b. Thus, although FIG. 4 corresponds to a combined laminate web having a cross-section such as illustrated in FIG. 2d, it will be understood that the combined laminate web c4 may be formed in accordance with any one of the variants of FIGS. 2d to 2b, or 2f, or the variants of FIGS. 6a and 6b.

As is clear to a person skilled in the art, the features and advantages as described in the above in relation to the exemplary combined panels 4 are equally applicable to a combined laminate web c4.

### Method for Forming a Combined Laminate Web

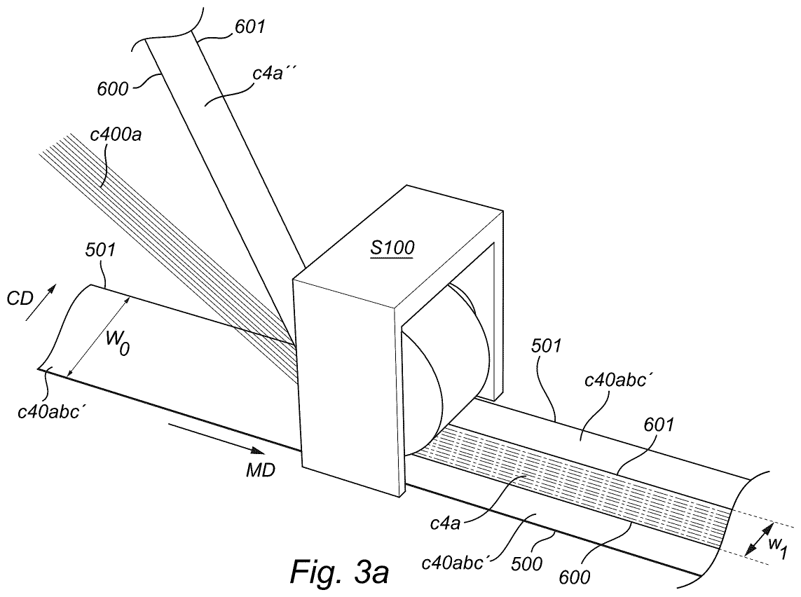

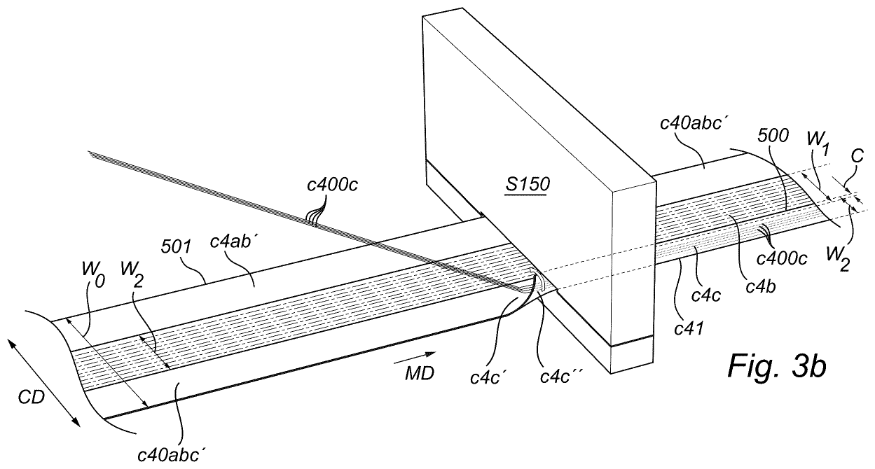

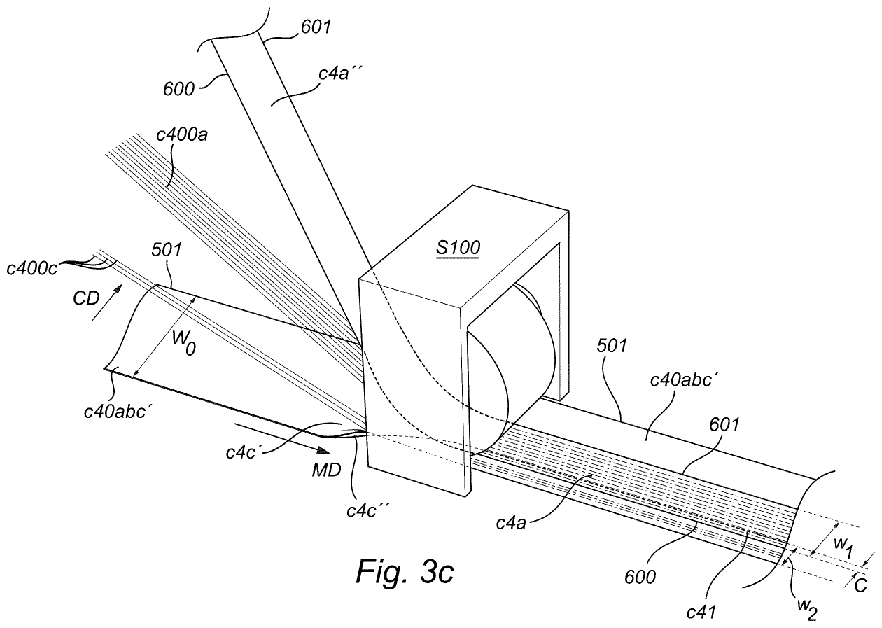

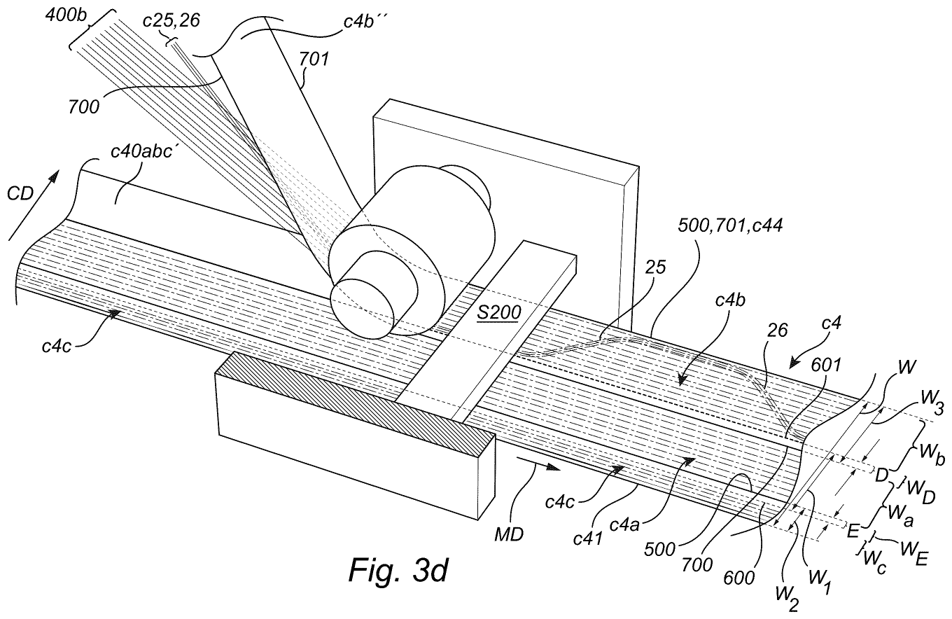

FIGS. 3a, 3b, and 3d together illustrate a first variant of a continuous method for forming a combined laminate web for a disposable hygienic article.

The illustrated variant of a method is for forming a combined laminate material web c4 as illustrated in FIG. 4, and with a cross-section as illustrated in FIG. 2b. This variant is believed to be advantageous from manufacturing point of view and for providing rear panels 4 with a uniform outer appearance.

However, features described in relation to this specific variant may be equally applicable to other variants of the method for forming a combined laminate material web, for example in accordance with any one of the cross-sections illustrated in FIG. 2d, 2e or 2f.

Thus, the method to be described is a method for forming a combined laminate material web c4 for a disposable hygienic article, the combined laminate material web being continuous as seen in a machine direction MD and having a width W between two side edges c41, c44 in a cross-direction CD perpendicular to the machine direction MD. The combined laminate material having a top side and a bottom side as seen in a thickness direction being perpendicular to the machine direction MD and to the cross-direction CD.

FIG. 3a schematically illustrates a welding bonding step S100 of the method. FIG. 3b shows a continuous combined bottom layer web c40ab′ which is being advanced in a machine direction MD. In the illustrated variant, the combined bottom layer web c40ab′ is to form both the first bottom layer 4a′ of the first web portion c4a and the second bottom layer 4b′ of the second web portion c4b of the combined web laminate c4 to be formed. Further, a continuous first top layer web c4a″ is being advanced in the machine direction MD. FIG. 3a also shows a first set of continuous elastic strands c400a being advanced in a machine direction MD so as to be sandwiched between the continuous combined bottom layer web c40abc′ and the continuous first top layer web c4a″.

The combined bottom layer web c40ab′ has a combined width WO in a direction cross the machine direction between first longitudinal edge 500 and a second longitudinal edge 501. The first top layer web c4a″ has a first width W1 in a direction cross the machine direction between a second and a first longitudinal edge 600, 601. As illustrated in the Figs., the first width W1 is less than the combined width WO.

In a welding bonding step S100, which is only schematically illustrated, the combined bottom layer web c40abc′ is bonded to the first top layer web c4a′ by a welded bond pattern, with the first set of elastomeric strands c400a being sandwiched between the combined bottom layer web c4bb′ and the first top layer web c4a′, so as to be aligned in parallel with each other and substantially with the machine direction.

As may be seen in FIG. 3a, in this variant the first top layer web c4a″ is bonded to the combined bottom layer web c40ab′ such that, in the cross-direction CD, a first portion of the combined bottom layer web c40ab′ extends beyond the first top layer web c4a″ towards the first side edge 500 of the combined bottom layer web c40abc′. As will be explained in the following, this first portion of the combined bottom layer web c40abc′ will be used to form a waist feature 50 of the combined laminate web c4. Thus, if selecting to use another form of waist feature 50, in other variants the first top layer web c4a″ could for example be arranged with a first longitudinal edge 600 coinciding with the first side edge 500 of the combined bottom layer web c40abc′.

Also, in this variant, the first top layer web c4a″ is bonded to the combined bottom layer web c40abc′ such that, in the cross-direction CD, a second portion of the combined bottom layer web c40abc′ extends beyond the first top layer web c4a″ towards the second side edge 500 of the combined bottom layer web c40abc′. As will be described in the following, this second portion of the combined bottom layer web c40abc′ will be used to form the second bottom layer 4b′ of the second web portion c4b of the combined laminate web c4.

FIG. 3b shows a variant of a waist forming step S150 for forming a waist laminate web portion c4c of the laminate web c4.

In this variant, the first portion as mentioned in the above of the combined bottom layer web c40abc′ is folded along the machine direction MD, such that the first portion forms, on each side of the fold, a waist bottom layer c4c′ and a waist top layer c4c″ in a waist web portion c4c of the combined laminate web 4.

As illustrated in FIG. 3b, a waist portion set of continuous elastic strands c400c is being advanced in a machine direction MD so as to be sandwiched between the waist bottom layer c4c′ and the waist top layer c4c″.

As illustrated in FIG. 3b, the folded portion forming the waist bottom layer c4c′ has a second width W2 in the cross-direction, extending from the fold to the first side edge 500 of the combined bottom layer web c40abc′. The second width W2 may be such that, in the connection region C, the waist top layer c4c″ overlaps with the first top layer c4a″.

The waist bottom layer c4c′ and the waist top layer c4c″ are bonded by a welding step S150 providing a welded bond pattern to attach the third set of elastic strands c400c between the waist bottom layer c4c′ and the waist top layer c4c″. As such, the waist forming step S150 may advantageously be performed upstream of the adhesive bonding step S200, which will be described in further detail in the below.

In the illustrated variant, the waist forming step S150 takes place downstream of the welding step S100 for forming the first web portion 4a. However, in other variants, the waist forming step S150 may take place upstream of the welding step S100. In such a variant, in the connection region C, the first top layer web c4a″ would be arranged to the top side of the waist top layer web c4c″.

FIG. 3d schematically illustrates an adhesive bonding step S200. FIG. 3c shows how a second top layer web c4b″ is fed in the machine direction MD together with a second set of continuous elastic strands c400b. The continuous elastic strands c400b of the second set are to be aligned in parallel with each other and substantially with the machine direction MD. Further, FIG. 3c shows how a set of one or more continuous additional elastic members c25, c26 are fed along the machine direction MD. The additional elastic members c25, c26 are to be positioned such that they extend at least partially in a direction not parallel with the machine direction. For example, and as illustrated in FIG. 3c, the additional elastic members c25, c26 may be arranged in an undulating pattern suitable for forming leg elastic members in body panels formed by the finalised combined laminate material web.

The second set of continuous elastic strands c400b and the additional elastic members c25, c26 are sandwiched between the second portion of the combined bottom layer web c40abc′ and the second top layer web c4b″, and adhesive bonds are applied so as to form the second laminate web portion c4b of the continuous laminate web material c4 in an adhesive bonding step S200.

The adhesive bonds may be applied to one or more out of the second top layer web c4b″ and the combined bottom layer web c40abc′, the second set of continuous elastic strands c400b, and the set of additional elastic members c25, c26.

For example, and as described herein in relation to FIG. 4, a first adhesive 80 may be applied substantially continuously to the second portion of the combined bottom layer web c40abc′ that is to be covered by the second top layer web c4b″. In addition, a second adhesive may be applied in defined regions 82 of the second top layer web c4b″ overlapping the additional elastic members c25, c26. Optionally, the second and first adhesives may be different, for example in that the second adhesive may be more fast curing than the first adhesive. Further, the adhesive bonding step may comprise applying adhesive to the second set of elastic strands c400b.

The second top layer web c4b″ has a third width W3 between a first longitudinal edge 700 and a second longitudinal edge 701. The second top layer web c4b″ may be arranged such that the second longitudinal edge 701 coincides with the second side edge 501 of the combined bottom layer web c40abc′. The third width W3 may be such that the second top layer web c4b″ overlaps with the first top layer web c4a″ in the connection region D.

Thus, a combined laminate web c4 is formed having a first web portion c4a having a width Wa, in this case corresponding to the first width W1 of the first top layer web c4a″ minus the width WD of the connection region D and the width WE of the waist connection region E. It is to be noted that the first web portion c4a encompasses the portions where the first top layer web c4a″ forms a top side surface and the first bottom layer web c4a′ forms a bottom side surface of the combined laminate web c4. The combined laminate web c4 further has a second web portion c4b having a width Wb in this case corresponding to the third width W3 of the second top layer web c4b″ minus the width WD of the connection region D. And finally, the combined laminate c4 comprises a waist web portion c4c having a width Wc corresponding the second width W2 of the folded waist top layer web c4c″ minus the width WE of the waist connection region E.

FIGS. 3a, 3c, and 3d together illustrate another variant of a method of forming a combined laminate web 4c, which in this case will have a cross-section such as illustrated in FIG. 2f.

In this method, the waist forming step S150 is performed simultaneously with the welding step S100 for forming the first web portion c4a. In practice, the waist forming step S150 and the welding step S100 is thus performed in a united welding step S100, S150 in the manufacturing line, using the same welding equipment.

Thus, in the united welding step S100, S150 which is only schematically illustrated, the combined bottom layer web c40abc′ is bonded to the first top layer web c4a″ by a welded bond pattern, with the first set of elastomeric strands c400a being sandwiched between the combined bottom layer web c4bb′ and the first top layer web c4a′, so as to be aligned in parallel with each other and substantially with the machine direction. Also, in the united welding step S100, 150 a first portion of the combined bottom layer web c40abc′ is folded along the machine direction MD, such that the first portion forms, on each side of the fold, a waist bottom layer c4c′ and a waist top layer c4c″ in a waist web portion c4c of the combined laminate web c4. A waist portion set of continuous elastic strands c400c is being advanced in a machine direction MD so as to be sandwiched between the waist bottom layer web c4c′ and the waist top layer web c4c″.

After the united welding step S100, S150 is performed, the adhesive bonding step S200 as described in relation to FIG. 3d in the above may be performed, so as to form the combined laminate material web c4.

In the first variant of the method as illustrated by FIGS. 3a, 3b and 3d, the waist top layer 4c″ is arranged to the top side of the first top layer 4a″. Thus, the first top layer web c4a″ is arranged adjacent the combined bottom layer web c40abc′, whereafter the combined bottom layer web c40abc′ is folded so as to form the waist portion c4c with the waist top layer c4c″ overlapping the first top layer web c4a″ in the waist connection region E. Thereafter, the united welding step S100, S150 is performed. The cross-section of the combined laminate web material c4 is as illustrated in FIG. 4d.

In the second variant of the method as illustrated by FIGS. 3a, 3c and 3d, the waist top layer 4c″ is arranged to the bottom side of the first top layer 4a″. Thus, the combined bottom layer web c40abc′ is first folded so as to form the waist portion c4c with the waist top layer c4c″, whereafter the first top layer web c4a″ is arranged adjacent the combined bottom layer web c40abc′, so as to overlap the waist top layer c4c″ is the waist connection region E. Thereafter, the united welding step S100, S150 is performed. The cross-section of the combined laminate web material c4 is as illustrated in FIG. 4f. However, the arrangement of the webs resulting in the different cross-sections as illustrated in FIGS. 4d and 4f may be selected as desired regardless of whether the welding is performed in a welding step 100 and a waist forming step 150 being performed as separate steps, or as a united step 100, 150.

In the welding bonding steps and the adhesive bonding step as described in the above, the steps are performed such that the respective set of elastomeric strands is attached between the respective top and bottom layers when in a stretched state in relation to the respective top and bottom layers. For example, the elastomeric strands may be stretched so as to be elongated to a length being at least 2× a relaxed length of the elastomeric strands, such as to a length being at least 3× a relaxed length of the elastomeric strands. The respective top and bottom layers may be stretched only slightly, i.e., to an amount required to perform the feeding operation. As such, the elastomeric strands are attached when in a stretched state in relation to the respective top and bottom layer. Accordingly, in a relaxed state of the combined laminate web, wherein the elastic strands are contracted, the top and bottom layers may form wrinkles along the elastic strands. The combined laminate web is stretchable along the machine direction from the relaxed state to a stretched state, in which the wrinkles of the top and bottom layers may be reduced or significantly completely flattened out. Thus, the required elasticity of the combined laminate web along the machine direction is achieved.

The combined laminate material web c4 may be subject to additional manufacturing steps before being fed to a method for forming an absorbent article.

For example, zones of reduced elasticity may be formed with regular intervals along the combined laminate material web c4. In particular, such zones of reduced elasticity may be formed where the absorbent assemblies 7 of the articles 100 to be formed are to overlap with the combined laminate material web c4. In zones of reduced elasticity, the elasticity along the machine direction provided by the sets of elastic strands is removed or significantly reduced.

To provide zones of reduced elasticity in the combined laminate material web c4, known methods involving for example the inactivation or removal of elastomeric strands in the zones. For example, the methods may involve the cutting or destruction of any elastomeric strands in the zones.

### Method for Forming Absorbent Articles

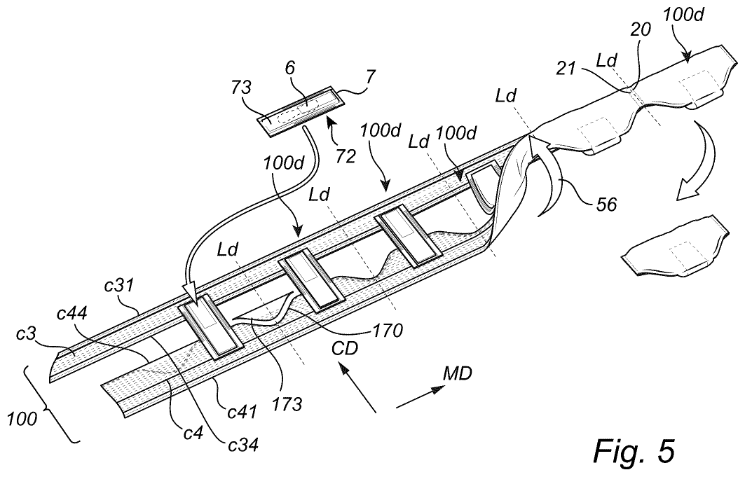

FIG. 5 schematically illustrates a method for producing a disposable pant-type article 1, such as an article as exemplified in the above.

The method comprises providing a rear body panel web c4 and a front body panel web c4, wherein at least one out of the rear body panel web c4 and the front body panel web c3 is a combined laminate material web c4c, for example as exemplified in the above.

As in the variant of FIG. 5, the method may comprise feeding a continuous front panel web c3 and a continuous rear panel web c4 in the machine direction MD, the front panel web c3 and the rear panel web c4 being spaced apart in the cross-direction Cd.

The front panel web c3 has a front waist edge c31 and an opposing front crotch edge c34. The rear panel web c4 has a rear waist edge c41 and an opposing rear crotch edge c44.

The rear panel web c4 is spaced apart from the front panel web c3 in a cross-direction CD, perpendicular to the machine direction MD, with a distance between the front panel web c3 and the rear panel web c4 in the cross-direction CD between the crotch edge c34 of the front panel web c3 and the crotch edge c44 of the rear panel web c4.

The front panel web c3 and the rear panel web c4 in the spaced apart condition together form a composite web 100 for forming interconnected precursor articles 100a-100e.

As illustrated in FIG. 5, the method further comprises applying and attaching absorbent assemblies 7 to the moving composite web 100 of interconnected precursor articles 100a-100e. Each absorbent assembly 7 comprises an absorbent core 6, a liquid impermeable backsheet 72 and a liquid permeable topsheet 73, the absorbent core 6 being sandwiched between the backsheet 72 and the topsheet 73.

Each absorbent assembly 7 is applied to the composite web 100 of precursor articles on a precursor article centerline extending in the cross-direction, CD.

The absorbent assemblies 7are applied spaced-apart in the machine direction, MD, and are attached with a front end part 7f of each absorbent assembly 7 overlapping a front panel zone of the front body panel web c3 and with a rear end part 7r of each absorbent assembly 7 overlapping a rear panel zone of the rear body panel web c4. The front panel zone and/or the rear panel zone may be provided with reduced elasticity, as mentioned in the above. For example, in the front panel zone and/or the rear panel zone, any elastic strands may be cut so as to disable the elasticity in the zones.

### Absorbent Core

The absorbent core 6 is disposed between the topsheet 73 and the backsheet 72 to absorb liquid, such as urine or other bodily fluids, which has passed through the topsheet 73. The topsheet 72, backsheet 73 and the absorbent core 6 may consist of any materials suitable for their purposes.

The absorbent core 6 may be a single-layer structure or may be a layered structure. The absorbent core 6 may comprise suitable amounts of superabsorbent material. Such superabsorbent material is well known in the field of absorbent articles and is constituted by water-swellable and water-insoluble material which is capable of absorbing large quantities of fluid upon formation of a hydrogel. The absorbent core 6 may contain superabsorbent material in the form of fibers or particles of absorbent polymer material. For example, the superabsorbent material may be surface cross-linked, partially neutralized polyacrylates. The superabsorbent material e.g., the superabsorbent fibers or particles, may be mixed with other absorbent or liquid uptake material or materials, such as cellulose fluff pulp, and/or be arranged in pockets or layers in the absorbent core 6.

The absorbent core 6 may further comprise components for improving properties of the absorbent core 6, such as integrity and strength. For example, the absorbent core 6 may comprise a binder or binders, such as binder fibers. Resilient fibers, chemically stiffened fibers, etc. may be present in the absorbent core 161 to counteract wet-collapse of cellulosic fibers. Such fibers may also be useful in retaining a fluid transporting capillary network in the absorbent component so that absorbent fluid may be distributed in the absorbent component and be absorbed by superabsorbent material also in parts of the absorbent component outside the initial wetting area of the absorbent article.

### Topsheet

The topsheet 73 is a wearer-facing layer and may comprise or consist of a fluid permeable nonwoven fabric, a perforated polymer film, mesh or foam. The topsheet may be made from thermoplastic material, such as thermoplastic synthetic fibers, film or netting. The topsheet 73 may consist of a single layer or may have a laminate structure comprising a plurality of layers, for example, two or more layers. The layers may be made of the same material, or some or all layers may be made of different materials.

### Backsheet

Furthermore, the backsheet 72 may be constituted by a liquid-impermeable layer such as a polymeric film, for example a film of polyethylene or polypropylene. The backsheet 72 may be breathable. The materials which may be used for the backsheet 72 include thin and flexible fluid impermeable plastic films, or fluid impermeable nonwoven materials, fluid impermeable foams and fluid impermeable laminates. The backsheet 72 may be formed by a single layer, or may be a multi-layered structure, with at least one layer being fluid impermeable.

### Leg Cuts

As illustrated in FIG. 5, after the application and attachment of an absorbent assembly 6 to the composite web 100 of interconnected precursor articles 100a-100e, leg cuts 170 may be formed in the composite web 100 of interconnected precursor articles 100a-100e between the precursor articles 100a-100e, each leg cut 170 having a curved configuration as indicated between the precursor articles 100d and 100e in FIG. 5. The leg cuts form a first leg edge 18 of the leading precursor article 100d and a second leg edge 19 of the trailing precursor article 100e.

After forming the leg cuts 170, the cut-out portions 173 are removed from the composite web 100 of interconnected precursor articles 100a-100e and are not further part of the process as disclosed herein.

After forming the leg cuts, the absorbent assemblies 6 will be located centrally between the first leg edge 18 and the second leg edge 19 of each precursor article 100a-100e in the composite web 100 of interconnected precursor articles.

As is shown in FIG. 5, when the leg edges 18, 19 have been formed, the composite web 100 of interconnected precursor articles 100a-100e is folded along a fold-line extending in the machine direction MD to bring the waist edge c31 of the front panel web c3 into alignment with the waist edge c41 of the rear panel web c41.

### Side Seams

A first side seam 20 and a second side seam 21 are formed in each precursor article 100a-100e along the dividing lines Ld between the precursor articles 100a-100e, from the aligned waist edges c31, c41 of the front and rear panel webs c3, c4, to the leg cuts 170.

The first side seam 20 of a leading precursor article 100a and the second side seam 21 of a trailing precursor article 100b may be formed as a single broad seal extending along the dividing line Ld, as shown in FIG. 5. When cutting away individual pant-type articles from the chassis web 100 of precursor articles 100a-100e by severing the composite web 100 of interconnected precursor articles 100a-100e along the dividing lines Ld, the single broad seal shown in FIG. 5 will at the same time be divided into the two side seams 120, 121 of the interconnected precursor articles 100a, 100b.

Alternatively, the side seams 120, 121 may be formed as individual seals placed on each side of the dividing line Ld.

The side seams may be formed by any method suitable for the purpose, such as by thermo-welding, ultrasonic welding or by means of adhesive. In case of adhesive side seals, the adhesive is generally applied before the chassis web is folded along the folding line Lv.

### Waist

With reference to FIG. 4, the front body panel web c3 and the rear body panel web c4 are each formed so as to comprise an integral waist panel portion 3c, 4c. As mentioned in the above, the front body panel web c3 and/or the rear body panel web c4 may comprise other types of waist features. For example, the front body panel web c3 and/or the rear bod panel web c4 may be provide with a waist feature in the form of a pre-fabricated band-shaped waist elastic member which may comprise one or more elastic elements such as bands, strings, elastic film, elastic nonwoven, etc. Such a prefabricated band-shaped waist elastic member may be applied to each of the front body panel web c3 and/or the rear body panel web c4 prior to application of the absorbent assemblies 6 to the precursor web 100, as illustrated in FIG. 5.

For example, a waist feature may be applied along an edge of the front panel web and/or the rear panel web before or after cutting the precursor web into individual articles.

It is also to be understood that waist elastic may be added at the waist edges of a composite web of precursor articles at any stage of the process up until severing of individual absorbent pant-type articles from the composite web of precursor articles.

The pant-type articles as disclosed herein may be of the kind shown in the Figures and having a three-part chassis with an absorbent assembly or crotch panel being connected to a front panel and which is connected to a rear panel, such that the absorbent assembly extends longitudinally to bridge between the front panel and the rear panel. Alternatively, the pant-type article may have a unitary chassis having a combined laminate web extending the full distance between the front panel waist edge and the rear panel waist edge, the combined laminate web constituting at least a part of each of the front panel and the rear panel and constituting a crotch web material in the crotch region of the pant-type article.

In such an alternative embodiment, a combined laminate web may be formed, which combined laminate web may form both the front body panel 3 and the rear body panel 4 of the absorbent article. In this case, the combined laminate web may comprise at least a front body panel section and a rear body panel section. Still such a combined laminate web may comprise at least a first web portion and a second web portion as defined in the above examples. After the application and attachment of an absorbent assembly 6 to such a composite web, leg cuts 170 may be formed in the composite web 100 of interconnected precursor articles 100a-100e between the precursor articles 100a-100e, each leg cut 170 having a closed loop configuration as indicated between the precursor articles 100b and 100c in FIG. 5. As such, the closed-loop leg cuts 170 may be formed so as to provide a first front leg edge and a first rear leg edge of the leading precursor article, and a second front leg edge and a second rear leg edge of the trailing precursor article.

However, methods involving a unitary chassis being formed by a combined laminate web may require more use of material than the three-part chassis solutions.

### Weld Bonding

Numerous techniques involving welded bond patterns are known which may be used for attaching the first set of elastomeric strands 400a between the first bottom layer 4a′ and the first top layer 4a″ in the first panel portion 4a/first web portion c4a.

For example, the welded bond pattern may comprise pairs of adjacent weld bonds, the adjacent weld bonds being provided on each side of an elastomeric strand, for attaching each of the first set of elastomeric strands 400a.

In some variants, such pairs of adjacent weld bonds are applied so as to retain the elastomeric strand primarily by friction and/or clamping the elastomeric strand.

For example, the pairs of adjacent weld bonds are arranged such that, each of the elastomeric strands is anchored in a passage defined between such a pair of adjacent weld bonds, wherein a distance between the adjacent weld bonds across the passage is smaller than the diameter of the elastomeric strand when in a non-tensioned state. The elastic strands are applied between the top and bottom layers in a stretched state and the top and bottom layers are bonded to each other while the elastic strands are held in the stretched state. In the stretched state, the elastic strands have a smaller diameter than in a relaxed or non-tensioned state. Thereby, the elastic strands will be mechanically trapped between the bonds in the pairs of adjacent bonds as the stretched elastic strands are allowed to relax and resume their non-tensioned diameter.

In some variants, the application of such pairs of adjacent weld bonds involves melting of at least one out of the top layer, the bottom layer and the elastic strands, such that the pairs of adjacent weld bonds also involve a fusion connection between the top and/or bottom layer and the elastic strand.

The bonding pattern may be formed by ultrasonic bonding or heat bonding, the bond elements being formed by locally heating the layers which are bonded together to cause thermoplastic material in the laminated layers to soften or melt and subsequently consolidate in the bonds.

Advantageously, the welded bond pattern comprises ultrasonic bonds. The welded bond pattern may for example be created using a bonding system such as described in e.g. EP 4025 412 A1 or EP 3 519 162.

The bond pattern may comprise a plurality of discrete bonds, the bonds being arranged in a regular bonding pattern. For example, the discrete bonds may form a pattern of uniformly distributed discrete bonds.

The bonding elements may be distributed over the body panel in the regular bonding pattern over the full area of the body panel, i.e., also in any zones with reduced elasticity. In other words, the bonding pattern is the same in the body panel zone with reduced elasticity as in the other parts of the body panel. Instead, the reduced elasticity may be accomplished as mentioned herein by e.g. cutting or destroying the elastic strands.

The bond pattern may comprise a plurality of discrete bonds. The bonding pattern may be uniform all over the first portion 4a of the rear body panel 4.

The bond pattern may comprise a plurality of discrete bonds being arranged for example along lines or curves. For example, the bond pattern may comprise a plurality of discrete bonds being arranged so as to form lines forming an angle with the longitudinal direction L/cross-direction CD. Alternatively, the bond pattern may comprise a plurality of discrete bonds being arranged so as to follow undulating lines.

### Adhesive Bonding

The second panel portion 4b is bonded by adhesive bonds, wherein the adhesive bonds are applied to one or more out of the second bottom layer 4b′, the second top layer 4b″, the second set of elastomeric strands 400b, and the leg elastic members 25, 26. To this end, prior known adhesive techniques may be used.

In FIG. 4, illustrating a combined laminate web, a variant of an adhesive pattern is shown. The same pattern may be applied to the article of FIG. 2a, although it is not shown here for reasons of clarity of the drawings.

As seen in FIG. 4, adhesive is substantially continuously applied over the surface of the second top layer 4b″ which is facing the second bottom layer 4b′, as illustrated by the dashed region 80.

In addition, adhesive is applied only in defined regions 82 of the surface of the second bottom layer 4b′ which is facing the second top layer 4b″. The defined regions 82 have a size and distribution so as to overlap the additional elastic members c25, c26 i.e. the leg elastic members 25, 26 in the article 1 in the second panel portion 4b. Finally, in the illustrated variant, also the second set of elastomeric strands 400b are provided with adhesive.

For example, a second adhesive may substantially continuously over the second top layer 4b″ and a first adhesive may be applied in the defined regions of the second bottom layer 4b′. For example, the second adhesive may be different from the first adhesive in that the second adhesive is more fast consolidating than the first adhesive. The first adhesive may be intended for attachment of elastic members and the second adhesive may be intended for lamination of webs.

### Materials

At least one, preferably each, out of the second bottom layer 4b′, the second top layer 4b″, the first bottom layer 4a′, the first top layer 4a″, and optionally the waist bottom layer 4c′, and the waist top layer 4c″ comprises or consists of a sheet of a nonwoven material.

The nonwoven material may contain thermoplastic fibers, preferably the nonwoven material contain at least 50% thermoplastic fibres and more preferably at least 80% thermoplastic fibres.

The nonwoven material layers or webs may be of any suitable nonwoven material such as spunbond, air laid, wet laid, carded, electro spun or meltblown nonwovens. The nonwoven material may be bonded by any suitable technique, such as by needling, hydroentangling, ultrasonic welding, or thermobonding.

The fibers of the nonwoven materials used herein may be man-made fibers, natural fibers or mixtures of man-made and natural fibers. Man-made fibers include mono-component, bi-component and multicomponent fibers of polymers such as polyolefins, polyesters, polyacrylates, etc., as well as regenerated fibers such as viscose fibers and modal fibers. Natural fibres are for instance cellulosic fibres such as pulp fibers, cotton fibers, flax, hemp, etc.

For layers that are bonded by ultrasonic bonding or heat bonding, the nonwoven materials which are used to form the layers should contain a sufficient amount of thermoplastic fibres to allow the bond elements to be formed. Examples of suitable thermoplastic polymers for use in the nonwoven webs as disclosed herein are polyethylene polypropylene and other polyolefin homopolymers and copolymers and polyesters. The weldable nonwoven webs may have a high content of thermoplastic component and preferably contain at least 50% thermoplastic fibres and more preferably at least 80% thermoplastic fibres.

The nonwoven materials may have a basis weight of from 8 to 26 gsm, such as from 10 to 22 gsm.

The elastic material in the elastomeric strands and/or the leg elastic members arranged may be any suitable elastic material such as natural or synthetic rubber, thermoplastic elastomers, such as thermoplastic polyurethane or styrene block co-polymers or elastane, also referred as to spandex polyurethane-polyurea copolymer. The elastic strands and/or the elastic elements of the leg elastic members may be of the elastane type that is available under the trade name “LYCRA”, but any suitable elastic thread may be used.

The threads may have a linear mass density, dtex, of about 80-1200 dtex.

Thus, the second set of elastomeric strands 400b and/or the first set of elastomeric strands 400a and/or optionally the third set of elastomeric strands 400c may have a linear mass density, dtex, of from 80 to 1200 dtex.

The number of elastomeric strands in each panel portion, and the longitudinal spacing between the strands in each panel portion, may be selected so as to provide the panel with suitable elasticity and a good fit.

For example, and as in the illustrated variants, the longitudinal spacing between the strands in the second set of elastomeric strands 400b in the second panel portion 4b may be no less than the longitudinal spacing between the strands in the first set of elastomeric strands 400a in the first panel portion 4a.

For example, the longitudinal spacing between the strands in the first set of elastomeric strands 400a of the first panel portion 4a may be from 2 to 20 mm, and/or the longitudinal spacing between the strands in the second set of elastomeric strands 400b may be 2 to 35 mm, and/or the longituidnal spacing between the strands in the third set of elastomeric strands may be 2 to 12 mm .. While examples of different variants have been described in the above with reference to the drawing, other variants and modifications will readily occur to those skilled in the art. For example, shapes and sizes of the absorbent articles are not limited to the shapes and sizes shown in the drawings.

1-55. (canceled)

56. A disposable pant-type article, the article having a longitudinal direction and a transverse direction, and a thickness direction perpendicular to the longitudinal direction and the transverse direction, wherein an inside of the article is towards the wearer when the article is worn, and an outside of the article is away from the wearer when the article is worn, the article comprising:
a front body panel,
a rear body panel, and
a crotch region being located between the front body panel and the rear body panel, wherein an absorbent assembly comprising an absorbent core is arranged with a central part of the absorbent core being located in the crotch region;
the front body panel being joined to the rear body panel by a first front panel side edge being joined to a first rear panel side edge in a first side seam, and a second front panel side edge being joined to a second rear panel side edge in a second side seam;
such that the article forms a waist opening, defined by a waist edge of the front panel and a waist edge of the rear panel, and a pair of leg openings, defined by a pair of leg edges of the front body panel and a pair of leg edges of the rear body panel,
wherein each of the front body panel and the rear body panel has a top side and a bottom side as seen in the thickness direction of the article, and
at least one of the front body panel and the rear body panel is a combined panel, and the combined panel comprises a pair of leg elastic members arranged adjacent the pair of leg edges of the combined panel, wherein each leg elastic member extends at least partially in a direction other than parallel with the transversal direction,
wherein the combined panel comprises, as seen along the longitudinal direction, a first panel portion and a second panel portion, the first panel portion and the second panel portion being connected by a connection region,
wherein the first panel portion extends from the connection region in the longitudinal direction towards the waist edge of the combined panel, and the second panel portion extends from the connection region in the longitudinal direction towards the pair of leg edges, the second panel portion comprising the pair of leg elastic members,
wherein the first panel portion comprises a first bottom layer and a first top layer being superimposed such that, in the first panel portion, the first bottom layer forms a bottom side surface of the combined panel and the first top layer forms a top side surface of the combined panel, the first panel portion further comprises a first set of elastomeric strands being sandwiched between the first bottom layer and the first top layer, the elastomeric strands being aligned substantially in parallel with each other and with the transversal direction, and being spaced apart from each other in the longitudinal direction, and
the first panel portion is bonded by the first bottom layer being bonded to the first top layer by a welded bond pattern, attaching the first set of elastomeric strands between the first bottom layer and the first top layer, and
the second panel portion comprises a second bottom layer and a second top layer being superimposed such that, in the second panel portion, the second bottom layer forms a bottom side surface and the second top layer forms a top side surface of the combined panel, wherein the pair of leg elastic members are sandwiched between the second bottom layer and the second top layer, and
the second panel portion is bonded by the second bottom layer being bonded to the second top layer by adhesive bonds, attaching the pair of leg elastic members between the second bottom layer and the second top layer.

57. The article according to claim 56, wherein the second panel portion comprises a second set of elastomeric strands being sandwiched between the second bottom layer and the second top layer, the elastomeric strands being aligned substantially in parallel with each other and with the transversal direction, and being spaced apart from each other in the longitudinal direction, and
the second panel portion is bonded by the second bottom layer being bonded to the second top layer by adhesive bonds, attaching the pair of leg elastic members and the second set of elastomeric strands between the second bottom layer and the second top layer.

58. The article according to claim 56, wherein the welded bond pattern of the first panel portion comprises pairs of adjacent weld bonds, the adjacent weld bonds in a pair being provided on each side of an elastomeric strand.

59. The article according to claim 56, wherein the first panel portion is free from adhesive.

60. The article according to claim 57, wherein the adhesive bonds of the second panel portion are applied to one or more of the second top layer, the second bottom layer, the second set of elastomeric strands, and the leg elastic members.

61. The article according to claim 57, wherein each leg elastic member crosses at least one elastic strand out of the second set of elastomeric strands.

62. The article according to claim 56, wherein the connection region has a length in the longitudinal direction being no more than 10%, of a total length of the combined panel in the longitudinal direction, and/or
wherein the first panel portion has a length in the longitudinal direction, the length being at least 20% of a total length of the combined panel in the longitudinal direction, and/or
wherein the second panel portion has a length in the longitudinal direction being at least 20% of a total length of the combined panel in the longitudinal direction.

63. The article according to claim 56, wherein the second bottom layer and the first bottom layer are formed by a single continuous material sheet, extending over the transversal connection region.

64. The article according to claim 56, wherein the second top layer and the first top layer are formed by separate material sheets.

65. The article according to claim 56, wherein the top side of the combined panel forms an inside of the article, and the bottom side of the combined panel forms an outside of the article.

66. The article according to claim 56, wherein the adhesive bonds of the second panel portion comprise, in defined regions overlapping the leg elastic members, a first adhesive applied to one of the second top layer and the second bottom layer, and a second adhesive applied to the other one of the second top layer and the second bottom layer, wherein optionally the first adhesive is different from the second adhesive.

67. The article according to claim 56, wherein the combined panel further comprises a waist panel portion comprising the waist edge of the combined panel.

68. The article according to claim 57, wherein the average longitudinal spacing between the strands in the second set of elastomeric strands of the second panel portion is no less than the average longitudinal spacing between the strands in the first set of elastomeric strands in the first panel portion.

69. The article according to claim 56, wherein the absorbent core is arranged with a central part of the absorbent core being located in the crotch region and with a front end part of the absorbent core overlapping with the front body panel in a front overlap area and with a rear end part of the absorbent core overlapping with the rear body panel in a rear overlap area.

70. The article according to claim 56, wherein the absorbent core is arranged so as to longitudinally overlap a transversal portion of the transversal connection region.

71. The article according to claim 56, wherein one of the front body panel and rear body panel is a combined panel, and the other of the front body panel and rear body panel is free from adhesive.

72. A method for forming a combined laminate material web for a disposable hygienic article, the combined laminate material web being continuous as seen in a machine direction and having a width in a cross-direction perpendicular to the machine direction, between a first side edge and a second side edge, the combined laminate material web defining a top side and a bottom side as seen in a thickness direction being perpendicular to the machine direction and to the cross-direction, the method comprising
a welding bonding step comprising
feeding a first bottom layer web, a first top layer web, and a first set of continuous elastic strands in the machine direction, and
bonding the first bottom layer web to the first top layer web by a welded bond pattern so as to attach the first set of continuous elastic strands between the first bottom layer web and the first top layer web, the first set of elastomeric strands being aligned substantially in parallel with each other and with the machine direction, and being spaced apart from each other in the cross-direction; and
an adhesive bonding step comprising:
feeding a second bottom layer web, and a second top layer web in the machine direction, and feeding a set of one or more continuous additional elastic members to be sandwiched between said second bottom layer web and second top layer web, and
bonding the second bottom layer web to the second top layer web by adhesive bonds so as to attach the set of one or more continuous additional elastic members, between the second bottom layer web and the second top layer web,
the additional elastic members extending at least partially in a direction not parallel with the machine direction, so as to form
a first web portion, wherein the first bottom layer web forms a bottom side surface and the first top layer web forms a top side surface of the combined laminate material web, and
a second web portion, wherein the second bottom layer web forms a bottom side surface and the second top layer web forms a top side surface of the combined laminate material web,
the second web portion being connected to the first web portion via a connection region extending along the machine direction.

73. The method according to claim 72, wherein the adhesive bonding step further comprises feeding a second set of continuous elastic strands in the machine direction to be sandwiched between said second bottom layer web and said second top layer web, and bonding the second bottom layer web to the second top layer web by adhesive bonds so as to attach the second set of elastomeric strands and the set of one or more continuous additional elastic members between the second bottom layer web and the second top layer web, the second set of elastomeric strands being aligned substantially in parallel with each other and with the machine direction, and being spaced apart from each other in the cross-direction.

74. The method according to claim 72, wherein the welding bonding step is performed upstream of the adhesive bonding step in the machine direction.

75. The method according to claim 73, wherein, in the adhesive bonding step, the elastomeric strands are aligned substantially in parallel with each other and with the machine direction, and the additional elastic members are extending at least partially in a direction not parallel with the machine direction, such that each one of the additional elastic members crosses at least one elastic strand out of the second set of elastomeric strands.

76. The method according to claim 72, wherein the welded bond pattern in the welding bonding step comprises pairs of adjacent weld bonds for attaching each of the first set of elastomeric strands using such pairs of adjacent weld bonds.

77. The method according to claim 73, wherein the adhesive bonds of the adhesive bonding step are applied to one or more out of the second bottom layer, the second top layer, the second set of elastomeric strands, and the leg elastic members.

78. The method according to claim 72, wherein the connection region has a width in the cross-direction being no more than 10% of the width of the combined laminate material web, and/or
wherein the second web portion has a width in the cross-direction being at least 20% of the width of the combined laminate material web, and/or
wherein the first web portion has a width in the cross-direction being at least 20% of the width of the combined laminate material web.

79. The method according to claim 72, wherein the welding bonding step comprises
feeding a combined bottom layer web material to form the first bottom layer web, and
bonding the first top layer web to a first width portion of the combined bottom layer web material so as to form the first web portion, leaving at least a second width portion of the combined bottom layer web material non-laminated, and
the adhesive bonding step comprises
feeding the combined bottom layer web material to form the second bottom layer web, and
bonding the second top layer web to the non-laminated second width portion of the combined bottom web so as to form the second web portion.

80. The method according to claim 72, wherein the second top layer web and the first top layer web are fed as separate web materials.

81. The method according to claim 72, wherein the adhesive bonding step comprises bonding the second top layer web in a partially overlapping relationship with the first top layer web, as seen along the cross-direction, such that the connection region comprises the overlapping portions of the second top layer web and the first top layer web.

82. The method according to claim 72, wherein the adhesive bonding step comprises, in defined regions overlapping the leg elastic members, applying a second adhesive to one of the second top layer web and the second bottom layer web, and applying a first adhesive to the other one of the second top layer web and the second bottom layer web, wherein optionally the second adhesive is different from the first adhesive.

83. The method according to claim 73, wherein the longitudinal spacing between the strands in the second set of elastomeric strands is no less than the longitudinal spacing between the strands in the first set of elastomeric strands.

84. The method according to claim 72, wherein the combined laminate material web is for forming a combined panel being a front body panel or a rear body panel of a disposable pant-like article, wherein the additional elastic members are applied so as to form leg elastic members in the combined panel.

85. A combined laminate material web for a disposable hygienic article, the combined laminate material web being continuous as seen in a machine direction and having a width in a cross-direction, perpendicular to the machine direction, between two side edges, the combined laminate material web having a top side and a bottom side as seen in a thickness direction perpendicular to the machine direction and to the cross-direction,
the combined laminate material web comprising a first web portion and a second web portion, the first web portion and the second web portion being connected in a connection region extending along the machine direction, such that the first web portion extends from the connection region and in the cross-direction towards the first side edge of the combined laminate material web, and the second web portion extends from the connection region and in the cross-direction towards the second side edge of the combined laminate material web,
the first web portion comprises a first bottom layer and a first top layer being superimposed such that, in the first web portion, the first bottom layer forms a bottom side surface and the first top layer forms a top side surface of the combined laminate material web,
the first web portion further comprising a first set of elastomeric strands being sandwiched between the first bottom layer and the first top layer, the elastomeric strands being aligned substantially in parallel with each other and with the machine direction, and the elastomeric strands being spaced apart from each other in the cross-direction, and
the first web portion is bonded by the first bottom layer being bonded to the first top layer by a welded bond pattern attaching the first set of elastomeric strands between the first bottom layer and the first top layer, and
the second web portion comprises a second bottom layer and a second top layer being superimposed such that, in the second web portion, the second bottom layer forms a bottom side surface and the second top layer forms a top side surface of the combined laminate material web,
the second web portion further comprising additional elastic members extending at least partially in a direction other than parallel with the machine direction, wherein the additional elastic members are sandwiched between the second bottom layer and the second top layer, and
the second web portion is bonded by the second bottom layer being bonded to the second top layer by adhesive bonds, attaching the additional elastic members between the second bottom layer and the second top layer.

86. A combined material web produced by the method of claim 72.

87. A method for producing a disposable pant-type article comprising
providing a combined laminate material web in accordance with claim 85,
forming a disposable pant-type article having a front body panel and a rear body panel, a crotch region being located between the front body panel and the rear body panel, and an absorbent assembly comprising an absorbent core being arranged with a central part of the absorbent assembly being located in the crotch region, wherein
a first front panel side edge being joined to a first rear panel side edge in a first side seam; and
a second front panel side edge being joined to a second rear panel side edge in a second side seam,
wherein at least one of the front body panel and rear body panel is formed from the combined laminate material web.

88. The method according to claim 87 for producing a disposable pant-type article comprising:
providing a rear body panel web and a front body panel web, at least one of the rear body panel web and the front body panel web being a combined laminate material web;
intermittently applying absorbent assemblies to the rear body panel web and the front body panel web, the absorbent assemblies being applied spaced-apart in the machine direction and being attached with a first end part of the core assembly overlapping the rear body panel web and a second end part of the core assembly overlapping the front body panel web, so as to form a composite web of interconnected precursor articles each precursor article comprising an absorbent assembly and the composite web of precursor articles having a front waist edge and a rear waist edge;
folding the composite web of interconnected precursor articles along a fold line extending in the machine direction and bringing the rear waist edge of the rear body panel web into alignment with the front waist edge of the front body panel web;
forming first and second side seams in each precursor article, the side seams joining the front body panel web with the rear body panel web and being formed in the cross-direction along dividing lines between the precursor articles; and
severing individual pant-type articles from the composite web of precursor articles.

89. The method according to claim 87, wherein the top side of the combined laminate material web forms the inside of the article and the bottom side of the combined laminate material web forms the outside of the article.

90. The method according to claim 87, comprising arranging the absorbent assembly with a central part of the absorbent assembly being located in the crotch region and with a front end part of the absorbent assembly overlapping with the front body panel in a front overlap area and with a rear end part of the absorbent assembly overlapping with the rear body panel in a rear overlap area.

91. The method according to claim 87, comprising arranging the absorbent assembly so as to overlap at least a part of the first web portion.
